(19) United States
(12) Patent Application Publication
(10) Pub. No.: US 2025/0114444 A1
(43) Pub. Date: Apr. 10, 2025

(54) COMPOSITIONS OF VIRUS-LIKE PARTICLES AND MICROCRYSTALLINE TYROSINE

(71) Applicant: UNIVERSITÄT BERN, Bern (CH)

(72) Inventors: Mona MOHSEN, Rämismühle (CH); Martin BACHMANN, Bern (CH)

(21) Appl. No.: 18/729,430

(22) PCT Filed: Jan. 18, 2023
(86) PCT No.: PCT/EP2023/051095

§ 371 Date: Jul. 16, 2024

(30) Foreign Application Priority Data
Jan. 19, 2022 (EP) 22152314.5

(51) Int. Cl. A61K 39/12; A61K 39/00; A61K 39/39; A61P 35/00

(52) U.S. Cl. CPC A61K 39/12; A61K 39/39; A61P 35/00; A61K 2039/5258; A61K 2039/55511; A61K 2039/585; A61K 2039/876; C12N 2770/14023; C12N 2770/14034; C12N 2770/14071

(57) ABSTRACT

The present invention relates to compositions comprising virus-like particles and microcrystalline tyrosine and its use for treating cancer.

The present invention relates to compositions comprising virus-like particles and microcrystalline tyrosine and its use for treating cancer.

### RELATED ART

Cancer immunotherapy has brought significant improvements for patients in terms of survival and quality of life in recent decades and has established itself as a pillar of cancer care (Esfahani K et al., Curr Oncol. 2020 April:27(S2)87-97).

Cancer vaccines, among them, have hereby been developed with the aim to avoid cancerogenic infections or destroy cancer cells in line with classical vaccination to train the immune system for blocking the offending pathogen. Great success has been achieved through the prophylactic cancer vaccines against human papilloma virus (HPV) responsible for cervical cancer, and against Hepatitis B virus (HBV) causing liver cancer (Stanley M., Philos Trans R Soc Lond B Biol Sci. 2017; 372(1732); Liu M A., Philos Trans R Soc Lond B Biol Sci. 2011; 366(1579):2823-6).

In contrast to prevention, development of vaccines against established cancers is more challenging, because tumors are often immunosuppressive and it is difficult to identify appropriate targets. Moreover, the so far used vaccines are of rather low immunogenicity (Hollingsworth R E, Jansen K., NPJ Vaccines. 2019; 4:7). Previous attempts to develop therapeutic cancer vaccines have mainly been based on targeting tumor-associated antigens (TAAs) and recently on targeting tumor-specific antigens (TSAs). Targeting TAAs has only shown modest effects likely related to immune tolerance to self-antigens. High affinity T cells directed against self-antigens are eliminated during T cell development, and additional peripheral tolerance mechanisms are responsible for keeping immune responses at low levels. In contrast, TSAs are either virally derived or mutated self-peptides and are not or less expected to have induced T cell tolerance. Nevertheless, the immune system may not respond well to these tumor antigens, and the immune system may become weakened due to side effects of cancer treatment, patient age and T cell exhaustion (Hollingsworth R E, Jansen K., NPJ Vaccines. 2019; 4:7).

The use of virus-like particles (VLPs) for vaccination against cancer has recently gained increased interest due to its possible combined induction of effective cellular and humoral immune responses (Mohsen M O et al, WIREs Nanomed Nanobiotechnol. 2019; e1579). In order to further increase the CTL responses when vaccinating with VLPs, the physiological properties of the lymphatic system was harnessed. Cucumber mosaic virus-like particles displaying the LCMV-gp33 peptide were formulated with the micron-sized microcrystalline adjuvant (MCT) showing not only that the CuMVTT-p33 nanoparticles decorate the surface of the micron-sized MCT adjuvant and form a local depot but vaccination with this combination enhanced the specific CTL response, mediating strong anti-tumor effects in the stringent B16F10p33 murine tumor model (Mohsen, Heath, et al., 2019; Heath M H et al, 2020, Frontiers in Immunology 11: 594911).

In situ (intratumoral) immunotherapy has also recently attracted interest in treating solid cancers, and aims at generating local and systemic anti-tumour effects (Marabelle A, et al., Ann Oncol. 2017; 28(suppl_12): xii33-xii43; Nobuoka D, et al., Hum Vaccin Immunother. 2013; 9(6):1234-6). In particular, the therapeutic accessibility of malignant melanoma lesions permits increased interest for developing in situ and local therapies (Middleton M R, et al., Br J Cancer. 2020; 123(6):885-97). Some recent examples of intratumoral treatment in melanoma includes CMP-001, a virus-like particle (VLP) loaded with A-type CpGs in combination with systemic anti-PD-1 (Ribas A, et al., Cancer Discov. 2021, 2998). Moreover, Lizotte et al. have shown that intratumoral injection of eCPMV is effective in treating dermal B16F10 melanomas, forming central tumor necrosis (Lizotte P H, et al. Nat Nanotechnol. 2016; 11(3):295-303). Another study has combined flexuous plant VLPs (PVX) with the chemotherapeutic drug Doxorubicin (DOX) either packaged into the VLPs or admixed together to treat B16F10 melanoma revealing that intra-tumoral injection of PVX-VLPs could delay tumour progression, and coadministration of the VLPs with DOX enhanced the induced anti-tumour response and increased survival (Lee K L, et al., Nano Lett. 2017; 17(7):4019-28).

In light of the still substantial challenges to improve immunotherapies against cancer, there is a need for effective, affordable, fast and safe immune therapies that can be widely used for different types of tumors, in particular solid tumors.

### SUMMARY OF THE INVENTION

We have surprisingly found that microcrystalline tyrosine (MCT) formulated and decorated with virus-like particles (VLPs) lead to robust and efficient anti-tumour responses, and inhibited local and distant tumor growth. Importantly hereby, the present inventive immunotherapy does not require the identification of patient-individual relevant antigens and, furthermore, the compositions of the present invention do not comprise tumor-associated or tumor-specific antigens associated or covalently linked to the VLP.

In particular, the preferred compositions of the present invention comprising microcrystalline tyrosine (MCT) formulated and decorated with virus-like particles (VLPs) were found to protect from murine melanoma and inhibit B16F10 tumor growth locally and systemically as shown in a B16F10 transplanted melanoma mice model. Furthermore, the inventive compositions when injected intratumorally formed immunogenic depots in injected tumors and enhanced polyfunctional CD8⁺ and CD4⁺ T cells and support the induction of systemic protection (from non-injected distant tumors) via re-circulating T cells. In addition, the local inflammation and immune response was associated with upregulation of genes involved in complement activation and collagen formation.

Thus, in a first aspect, the present invention provides for a composition comprising particles, wherein each particle comprises, preferably consists of,
(a) at least one virus-like particle (VLP), preferably a recombinant VLP, and
(b) at least one microcrystalline tyrosine, preferably microcrystalline L-tyrosine, wherein said microcrystalline tyrosine has a median particle size of and between 0.1 μm and 50 μm, preferably of and between 1 μm and 10 μm, as preferably determined by Flow Particle Image Analyser (FPIA) and further preferably as described in Example 2; and
wherein said VLP does not comprise a tumor-associated antigen or a tumor-specific antigen covalently linked to said VLP, and wherein further preferably said VLP does not comprise an antigen (such as an allergen) covalently linked to said VLP.
Preferably said composition is capable of treating tumors, preferably malignant tumors, and further preferably is capable of treating solid tumors, preferably malignant solid tumors, and again further preferably said composition is capable of intratumorally treating malignant solid tumors.

In a further aspect, the present invention provides for a composition comprising, preferably consisting of,
(a) at least one virus-like particle (VLP), preferably a recombinant VLP, and
(b) at least one microcrystalline tyrosine, preferably microcrystalline L-tyrosine, wherein said microcrystalline tyrosine has a median particle size of and between 0.1 μm and 50 μm, preferably of and between 1 μm and 10 μm, as preferably determined by Flow Particle Image Analyser (FPIA) and further preferably as described in Example 2; and
wherein said VLP does not comprise a tumor-associated antigen or a tumor-specific antigen covalently linked to said VLP, and wherein further preferably said VLP does not comprise an antigen (such as an allergen) covalently linked to said VLP.
Preferably said composition is capable of treating tumors, preferably malignant tumors, and further preferably is capable of treating solid tumors, preferably malignant solid tumors, and again further preferably said composition is capable of intratumorally treating malignant solid tumors.

In again further aspect, the present invention provides for a composition comprising particles, wherein each particle comprises, preferably consists of,
(a) at least one virus-like particle (VLP), preferably a recombinant VLP, and
(b) at least one microcrystalline tyrosine, preferably microcrystalline L-tyrosine, wherein said microcrystalline tyrosine has a median particle size of and between 0.1 μm and 50 μm, preferably of and between 1 μm and 10 μm, as preferably determined by Flow Particle Image Analyser (FPIA) and further preferably as described in Example 2; and
wherein said VLP does not comprise a tumor-associated antigen covalently linked to said VLP, and wherein further preferably said VLP does not comprise an antigen (such as an allergen) covalently linked to said VLP.
Preferably said composition is capable of treating tumors, preferably malignant tumors, and further preferably is capable of treating solid tumors, preferably malignant solid tumors, and again further preferably said composition is capable of intratumorally treating malignant solid tumors.

In again further aspect, the present invention provides for a composition comprising, preferably consisting of,
(a) at least one virus-like particle (VLP), preferably a recombinant VLP, and
(b) at least one microcrystalline tyrosine, preferably microcrystalline L-tyrosine, wherein said microcrystalline tyrosine has a median particle size of and between 0.1 μm and 50 μm, preferably of and between 1 μm and 10 μm, as preferably determined by Flow Particle Image Analyser (FPIA) and further preferably as described in Example 2; and
wherein said VLP does not comprise a tumor-associated antigen covalently linked to said VLP, and wherein further preferably said VLP does not comprise an antigen (such as an allergen) covalently linked to said VLP.
Preferably said composition is capable of treating tumors, preferably malignant tumors, and further preferably is capable of treating solid tumors, preferably malignant solid tumors, and again further preferably said composition is capable of intratumorally treating malignant solid tumors.

Further aspects and embodiments of the present invention will be become apparent as this description continues.

### DESCRIPTION OF FIGURES

FIG. 1A: A sketch of the challenging therapeutic murine melanoma model.

FIG. 1B: Vaccination regimen and groups using intratumor injections.

FIG. 1C: Percentage of CD11b^{Hi} Ly6C^{Hi} (mean ±) SEM in blood on day 12 of mice transplanted with B16F10 melanoma tumor in each group.

FIG. 1D: Tumor volume mm³ (mean±SEM) measured on day 14 post-tumor transplantation for the groups treated with: PBS, CuMVtt-VLPs, or CuMVtt-VLPs formulated with MCT, each dot represents a tumor.

FIG. 1E: Density of CD8+ T cells (mean±SEM) in tumor, pre-gated on TILs (Tumor-infiltrating lymphocytes). The density was measured by dividing the total number of CD8+ T cells in each tumor by its volume.

FIG. 1F: Correlation between density of CD8+ T cells and tumor volume mm³.

FIG. 1G: Representative FACS plot showing the total number of CD8+ T cells in tumors, pre-gated on TILs. Statistical analysis by Student's t-test, (n=11 in the control group and n=10 in the other groups).

FIG. 1H: Density of CD8+ IFN-γ+ T cells (mean±SEM) in tumor, pre-gated on TILs. The density was measured by dividing the total number of CD8+ IFN-γ+ T cells in each tumor by its volume. For (n=5). One representative of 2 experiments is shown. *P<0.05: **P<0.01: ***P<0.0001

FIG. 1I: Representative FACS plot showing the total number of CD8+ IFN-γ+ producing cells, pre-gated on TILs.

FIG. 2A: Total IgG antibodies against CuMVtt-VLPs in the sera from mice treated intratumorally with PBS, CuMVtt-VLPs, or CuMVtt-VLPs formulated with MCT measured at OD450.

FIG. 2B: 1:20 dilution of total IgG antibodies against CuMVtt-VLPs in the sera from mice treated intratumorally with PBS, CuMVtt-VLPs, or CuMVtt-VLPs formulated with MCT measured at OD450. statistical analysis by Student's t-test, (n=5). One representative of 2 experiments is shown. *P<0.05: **P<0.01: ***P<0.0001.

FIG. 3A: Control group treated with PBS: Necrosis comprises 30% of the tumor section. Necrosis is indicated as grey areas with white lining (*).

FIG. 3B: Group treated with CuMVtt-VLPs+MCT: Necrosis comprises 2% of the tumor section. Necrosis is indicated as grey areas with white lining (*).

FIG. 3C: Control group treated with PBS: characterized by the highest TMR (TMR). Each white dot corresponds to a mitotic figure. Note that only the left (approximately 60%) of the tumor was assessed for the TMR.

FIG. 3D: Group treated with CuMVtt-VLPs+MCT: Tumor area 17 mm2, mitotic rate (TMR) of 47/mm2. Each white dot corresponds to a mitotic figure. The entire tumor cross section was assessed for the TMR. H&E staining.

FIG. 4A: Experiment plan.

FIG. 4B: % of CD8+ T cells in blood of naïve C57/BL/6 mice before and following CD8+ T cells depletion using anti-CD8a mAb, depletion efficacy is about 95%.

FIG. 4C: Representative FACS plot showing CD8+ T cell population before and after depletion.

FIG. 4D: % of CD8+ T cells in blood of C57/BL/6 mice bearing B16F10 tumor and treated with intratumor injection of PBS, CuMVtt-VLPs+MCT or CuMVtt-VLPs+MCT (after CD8+ T cell depletion).

FIG. 4E: Tumor volume mm³ (mean+/−SEM) measured on day 14 post-tumor transplantation for the groups treated with: PBS, CuMVtt-VLPs+MCT or CuMVtt-VLPs+MCT (after CD8+ T cell depletion). Each dot represents a tumor, statistical analysis by Student's t-test, (n=5). One representative of 2 experiments is shown. *P<0.05: **P<0.01: ***P<0.0001.

FIG. 5A: A sketch of the challenging therapeutic murine melanoma model for abscopal effect experiment including vaccination regimen and groups using intratumor injections.

FIG. 5B: Tumor volume mm³ (mean±SEM) for right and left tumors measured on day 14 post-tumor transplantation for the groups treated with: PBS or CuMVtt-VLPs formulated with MCT, each dot represents a tumor.

FIG. 5C: Representative right (treated) and left (untreated) mice harboring B16F10 melanoma tumors on day 14.

FIG. 5D: Density of CD8+ T cells (mean±SEM) in right and left tumors, pre-gated on TILs (Tumor-infiltrating lymphocytes). The density was measured by dividing the total number of CD8+ T cells in each tumor by its volume.

FIG. 5E: Representative FACS plot showing the total number of CD8+ T cells in right and left tumors, pre-gated on TILs. Statistical analysis by Student's t-test, (n=5). One representative of 2 experiments is shown. *P<0.05: **P<0.01: ***P<0.0001.

FIG. 6: Tumor volume mm³ (mean±SEM) measured on day 14 post-tumor transplantation for the groups treated with: PBS, Qb-VLPs formulated with MCT, CuMVtt-VLPs formulated with MCT, or CCMVtt-VLPs formulated with MCT, each dot represents a tumor. Due to internal variation with each group and due to low number samples, we have combined the different VLPs+MCT and compare it to PBS group. Statistical analysis by Student's t-test, (n=5). One representative experiment is shown. *P<0.05: **P<0.01: ***P<0.0001.

FIG. 7A: Surgical dissection of the preputial melanoma mass of a grey stallion including amputation of the male genital organ.

FIG. 7B: External hard melanoma lesions on the lower part of the tail, circled.

FIG. 7C: 8 days post intratumor injection of the novel immune-enhancer; notice the softening of the lesion and the black melanoma discharge.

FIG. 7D: Melanoma lesion 2 days post the 3rd intratumor injection; notice the detachment of the solid tumor.

FIG. 7E: Melanoma lesion 2 days post the 3rd intratumor injection; notice the detachment of the solid tumor.

FIG. 7F: The same lesion 1-year post treatment, no progression of melanoma has been recorded.

FIG. 7G: Untreated melanoma nodules in the upper part of the tail, pointed with arrows.

FIG. 7H: The untreated nodules disappeared 1-year later.

FIG. 7I: Ultrasound image of untreated mammary gland lump in a grey mare, measures 4.61 cm×6.12 cm.

FIG. 7J: Ultrasound image of the mammary gland lump after 1 dose of intratumor-guided injection of the novel immune-enhancer measures 3.02 cm×5.07 cm.

### DETAILED DESCRIPTION OF THE INVENTION

Unless defined otherwise, all technical and scientific terms used herein have the same meanings as commonly understood by one of ordinary skill in the art to which this invention belongs.

The term “about”, as used herein shall have the meaning of +/−10%. For example about 50% shall mean 45% to 55%. Preferably, the term “about”, as used herein shall have the meaning of +/−5%. For example about 50% shall mean 47.5% to 52.5%.

The phrase “between number X and number Y”, as used herein, shall refer to include the number X and the number Y. For example, the phrase “between 0.1 μm and 50 μm” refers to 0.1 μm and 50 μm and the values in between. The same applies to the phrase “between about number X and about number Y”.

The term “% (w/v)” as used herein refers to (mass of the component/total volume of the composition)×100. By way of example, 4 wt % L-tyrosine refers to 4 g L-tyrosine per 100 ml of the composition.

When the terms “a,” or “an” are used herein, they mean “at least one” unless indicated otherwise.

Virus-like particle (VLP): The term “virus-like particle (VLP)” as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. A virus-like particle in accordance with the invention is non-replicative and non-infectious since it lacks all or part of the viral genome or genome function. A virus-like particle in accordance with the invention may contain nucleic acid distinct from their genome. Recombinantly produced virus-like particles typically contain host cell derived RNA. A typical and preferred embodiment of a virus-like particle in accordance with the present invention is a viral capsid composed of polypeptides of the invention. A virus-like particle is typically a macromolecular assembly composed of viral coat protein which typically comprises 60, 120, 180, 240, 300, 360, or more than 360 protein subunits per virus-like particle. Typically and preferably, the interactions of these subunits lead to the formation of viral capsid or viral-capsid like structure with an inherent repetitive organization. One feature of a virus-like particle is its highly ordered and repetitive arrangement of its subunits.

Virus-like particle of an RNA bacteriophage: As used herein, the term “virus-like particle of an RNA bacteriophage” refers to a virus-like particle comprising, or preferably consisting essentially of or consisting of coat proteins, mutants or fragments thereof, of an RNA bacteriophage. In addition, virus-like particle of an RNA bacteriophage resembling the structure of an RNA bacteriophage, being non replicative and/or non-infectious, and lacking at least the gene or genes encoding for the replication machinery of the RNA bacteriophage, and typically also lacking the gene or genes encoding the protein or proteins responsible for viral attachment to or entry into the host. Also included are virus-like particles of RNA bacteriophages, in which the aforementioned gene or genes are still present but inactive, and, therefore, also leading to non-replicative and/or non-infectious virus-like particles of an RNA bacteriophage. Preferred VLPs derived from RNA bacteriophages exhibit icosahedral symmetry and consist of 180 subunits (monomers). Preferred methods to render a virus-like particle of an RNA bacteriophage non replicative and/or non-infectious is by physical, chemical inactivation, such as UV irradiation, formaldehyde treatment, typically and preferably by genetic manipulation.

Virus-like particle of CuMV: The terms “virus-like particle of CuMV” or CuMV VLPs refer to a virus-like particle comprising, or preferably consisting essentially of, or preferably consisting of at least one CuMV polypeptide. Preferably, a virus-like particle of CuMV comprises said CuMV polypeptide as the major, and even more preferably as the sole protein component of the capsid structure. Typically and preferably, virus-like particles of CuMV resemble the structure of the capsid of CuMV. Virus-like particles of CuMV are non-replicative and/or non-infectious, and lack at least the gene or genes encoding for the replication machinery of the CuMV, and typically also lack the gene or genes encoding the protein or proteins responsible for viral attachment to or entry into the host. This definition includes also virus-like particles in which the aforementioned gene or genes are still present but inactive. Preferred methods to render a virus-like particle of CuMV non replicative and/or non-infectious is by physical or chemical inactivation, such as UV irradiation, formaldehyde treatment. Preferably, VLPs of CuMV lack the gene or genes encoding for the replication machinery of the CuMV, and also lack the gene or genes encoding the protein or proteins responsible for viral attachment to or entry into the host. Again more preferably, non-replicative and/or non-infectious virus-like particles are obtained by recombinant gene technology. Recombinantly produced virus-like particles of CuMV according to the invention typically and preferably do not comprise the viral genome. Virus-like particles comprising more than one species of polypeptides, often referred to as mosaic VLPs are also encompassed by the invention. Thus, in one embodiment, the virus-like particle according to the invention comprises at least two different species of polypeptides, wherein at least one of said species of polypeptides is a CuMV polypeptide. Preferably, a VLP of CuMV is a macromolecular assembly composed of CuMV coat protein which typically comprises 180 coat protein subunits per VLP. Typically and preferably, a VLP of CuMV as used herein, comprises, essentially consists of, or alternatively consists of, at least one CuMV polypeptide comprising or preferably consisting of (i) an amino acid sequence of a coat protein of CuMV; or (ii) a mutated amino acid sequence, wherein the amino acid sequence to be mutated is an amino acid sequence of a coat protein of CuMV, and wherein said mutated amino acid sequence and said amino acid sequence to be mutated show a sequence identity of at least 90%, preferably of at least 95%, further preferably of at least 98% and again more preferably of at least 99%.

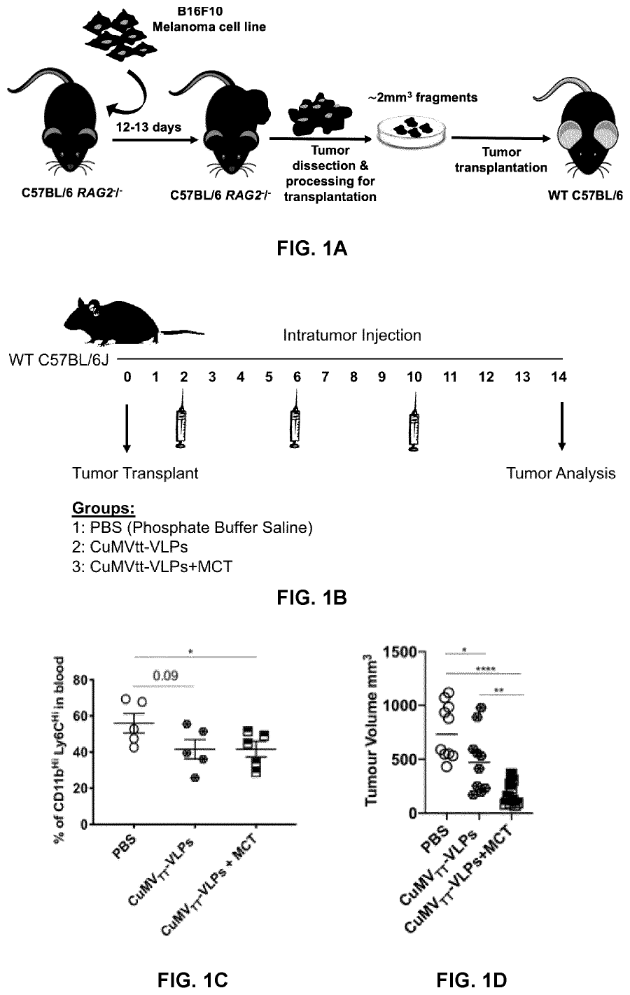

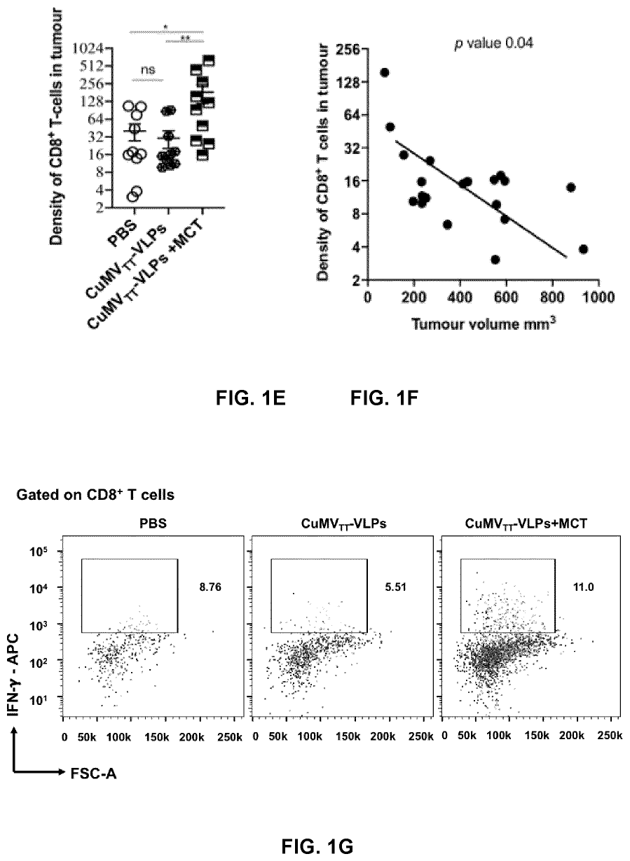

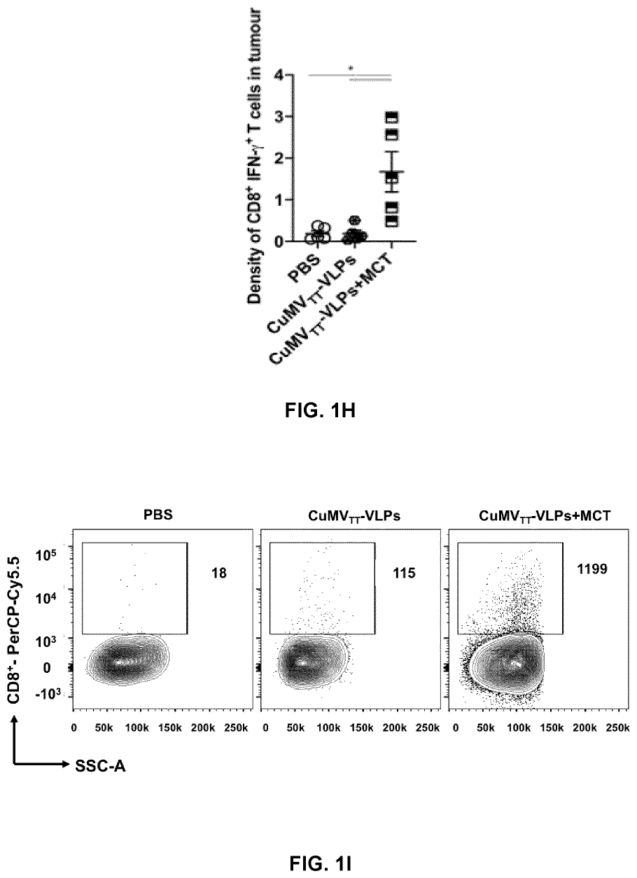

Tumor-associated antigen (TAA)/Tumor-specific antigen (TSA): The terms “tumor-associated antigen (TAA)” and “tumor-specific antigen (TSA)” are known to the skilled in the art. They are used herein as defined and further described in Hollingsworth R E, Jansen K., NPJ Vaccines. 2019; 4:7 (it is in particular referred to FIG. 1).

Solid Tumor: Solid tumors can be defined as abnormal mass of solid tissue. They can be benign or malignant and are named according to the cell type of origin. Examples of malignant tumors are carcinomas (derived from epithelial cells), sarcomas (derived from mesenchymal cells) or lymphomas (derived from lymphocytes). In contrast, so-called “liquid tumors” (e.g. leukemias) do not form solid tumors. Thus, solid malignant tumors and cancers, respectively, are defined as abnormal cellular growths in “solid” organs such as the breast or prostate, as opposed to leukemia, a cancer affecting the blood, which is liquid. The terms “solid cancer” and “solid malignant tumor” are interchangeably used herein.

Antigen: As used herein, the term “antigen” refers to a molecule capable of being bound by an antibody or a T-cell receptor (TCR) if presented by MHC molecules. An antigen is additionally capable of being recognized by the immune system and/or being capable of inducing a humoral immune response and/or cellular immune response leading to the activation of B- and/or T-lymphocytes. However, the term “antigen” as used herein does not refer to the virus-like particle contained in the inventive compositions and/or pharmaceutical compositions nor does it refer to a Th cell epitope included in modified polypeptides capable of assembling and forming said VLP. Moreover, the term “antigen” also does not refer to any component forming the particle or the composition of the invention, such as, for example the coat protein or parts thereof Coat protein: The term “coat protein” refers to a viral protein, preferably to a subunit of a natural capsid of a virus, preferably of an RNA bacteriophage or a plant virus, which is capable of being incorporated into a virus capsid or a VLP. The term coat protein encompasses naturally occurring coat protein as well as recombinantly expressed coat protein. Further encompassed are mutants and fragments of coat protein, wherein said mutants and fragments retains the capability of forming a VLP.

Polypeptide: The term “polypeptide” as used herein refers to a polymer composed of amino acid monomers which are linearly linked by peptide bonds (also known as amide bonds). The term polypeptide refers to a consecutive chain of amino acids and does not refer to a specific length of the product. Thus, peptides, and proteins are included within the definition of polypeptide.

Cucumber Mosaic Virus (CuMV) polypeptide: The term “cucumber mosaic virus (CuMV) polypeptide” as used herein refers to a polypeptide comprising or preferably consisting of: (i) an amino acid sequence of a coat protein of cucumber mosaic virus (CuMV), or (ii) a mutated amino acid sequence, wherein the amino acid sequence to be mutated is an amino acid sequence of a coat protein of CuMV, and wherein said mutated amino acid sequence and said amino acid sequence to be mutated, i.e. said coat protein of CuMV, show a sequence identity of at least 90%, preferably of at least 95%, further preferably of at least 98% and again more preferably of at least 99%. Typically and preferably, the CuMV polypeptide is capable of forming a virus-like particle of CuMV upon expression by self-assembly.

Coat protein (CP) of cucumber mosaic virus (CuMV): The term “coat protein (CP) of cucumber mosaic virus (CuMV)”, as used herein, refers to a coat protein of the cucumber mosaic virus which occurs in nature. Due to extremely wide host range of the cucumber mosaic virus, a lot of different strains and isolates of CuMV are known and the sequences of the coat proteins of said strains and isolates have been determined and are, thus, known to the skilled person in the art as well. The sequences of said coat proteins (CPs) of CuMV are described in and retrievable from the known databases such as Genbank, www.dpvweb.net, or www.ncbi.nlm.nih.gov/protein/. Examples are described in EP Application No. 14189897.3. Further examples of CuMV coat proteins are provided in SEQ ID NOs 1, 10, and 13. It is noteworthy that these strains and isolates have highly similar coat protein sequences at different protein domains, including the N-terminus of the coat protein. In particular, 98.1% of all completely sequenced CuMV isolates share more than 85% sequence identity within the first 28 amino acids of their coat protein sequence, and still 79.5% of all completely sequenced CuMV isolates share more than 90% sequence identity within the first 28 amino acids of their coat protein sequence. Typically and preferably, the coat protein of CuMV used for the present invention is capable of forming a virus-like particle of CuMV upon expression by self-assembly. Preferably, the coat protein of CuMV used for the present invention is capable of forming a virus-like particle of CuMV upon expression by self-assembly in E. coli.

Modified virus-like particle (VLP) of cucumber mosaic virus (CuMV): The term “modified virus-like particle (VLP) of cucumber mosaic virus (CuMV)” as used herein, refers to a VLP of CuMV which is a modified one in such as it comprises, or preferably consists essentially of, or preferably consists of at least one modified CuMV polypeptide, wherein said modified CuMV polypeptide comprises, or preferably consists of, a CuMV polypeptide, and a T helper cell epitope. Typically and preferably, said T helper cell epitope (i) is fused to the N-terminus of said CuMV polypeptide, (ii) is fused to the C-terminus of said CuMV polypeptide, (iii) replaces a region of consecutive amino acids of said CuMV polypeptide, wherein the sequence identity between said replaced region of consecutive amino acids of said CuMV polypeptide and the T helper cell epitope is at least 15%, preferably at least 20%, or (iv) replaces a N-terminal region of said CuMV polypeptide, and wherein said replaced N-terminal region of said CuMV polypeptide consists of 5 to 15 consecutive amino acids. Preferably, said T helper cell epitope replaces a N-terminal region of said CuMV polypeptide, and wherein said replaced N-terminal region of said CuMV polypeptide consists of 5 to 15 consecutive amino acids, preferably of 9 to 14 consecutive amino acids, more preferably of 11 to 13 consecutive amino acids, and most preferably of 11, 12 or 13 consecutive amino acids. Preferably said modified VLP of CuMV of the present invention is a recombinant modified VLP of CuMV.

Modified CuMV polypeptide: The term “modified CuMV polypeptide” as used herein refers to a CuMV polypeptide modified in such as defined herein, that said modified CuMV polypeptide comprises, or preferably consists of, a CuMV polypeptide, and a T helper cell epitope. Typically, the modified CuMV polypeptide is capable of forming a virus-like particle of CuMV upon expression by self-assembly. Preferably, the modified CuMV polypeptide is a recombinant modified CuMV polypeptide and is capable of forming a virus-like particle of CuMV upon expression by self-assembly in E. coli.

N-terminal region of the CuMV polypeptide: The term “N-terminal region of the CuMV polypeptide” as used herein, refers either to the N-terminus of said CuMV polypeptide, and in particular to the N-terminus of a coat protein of CuMV, or to the region of the N-terminus of said CuMV polypeptide or said coat protein of CuMV but starting with the second amino acid of the N-terminus of said CuMV polypeptide or said coat protein of CuMV if said CuMV polypeptide or said coat protein comprises a N-terminal methionine residue. Preferably, in case said CuMV polypeptide or said coat protein comprises a N-terminal methionine residue, from a practical point of view, the start-codon encoding methionine will usually be deleted and added to the N-terminus of the Th cell epitope. Further preferably, one, two or three additional amino acids, preferably one amino acid, may be optionally inserted between the stating methionine and the Th cell epitope for cloning purposes. The term “N-terminal region of the mutated amino acid sequence of a CuMV polypeptide or a CuMV coat protein” as used herein, refers either to the N-terminus of said mutated amino acid sequence of said CuMV polypeptide or said coat protein of CuMV, or to the region of the N-terminus of said mutated amino acid sequence of said CuMV polypeptide or said coat protein of CuMV but starting with the second amino acid of the N-terminus of said mutated amino acid sequence of said CuMV polypeptide or said coat protein of CuMV if said mutated amino acid sequence comprises a N-terminal methionine residue. Preferably, in case said CuMV polypeptide or said coat protein comprises a N-terminal methionine residue, from a practical point of view, the start-codon encoding methionine will usually be deleted and added to the N-terminus of the Th cell epitope. Further preferably, one, two or three additional amino acids, preferably one amino acid, may be optionally inserted between the stating methionine and the Th cell epitope for cloning purposes.

Recombinant polypeptide: In the context of the invention the term “recombinant polypeptide” refers to a polypeptide which is obtained by a process which comprises at least one step of recombinant DNA technology. Typically and preferably, a recombinant polypeptide is produced in a prokaryotic expression system. It is apparent for the artisan that recombinantly produced polypeptides which are expressed in a prokaryotic expression system such as E. coli may comprise an N-terminal methionine residue. The N-terminal methionine residue is typically cleaved off the recombinant polypeptide in the expression host during the maturation of the recombinant polypeptide. However, the cleavage of the N-terminal methionine may be incomplete. Thus, a preparation of a recombinant polypeptide may comprise a mixture of otherwise identical polypeptides with and without an N-terminal methionine residue. Typically and preferably, a preparation of a recombinant polypeptide comprises less than 10%, more preferably less than 5%, and still more preferably less than 1% recombinant polypeptide with an N-terminal methionine residue.

Recombinant CuMV polypeptide: The term “recombinant CuMV polypeptide” refers to a CuMV polypeptide as defined above which is obtained by a process which comprises at least one step of recombinant DNA technology. Typically and preferably a preparation of a recombinant CuMV polypeptide comprises less than 10%, more preferably less than 5%, and still more preferably less than 1% recombinant CuMV polypeptide with an N-terminal methionine residue. Consequently, a recombinant virus-like particle of the invention may comprise otherwise identical recombinant polypeptides with and without an N-terminal methionine residue.

Recombinant modified CuMV polypeptide: The term “recombinant modified CuMV polypeptide” refers to a modified CuMV polypeptide as defined above which is obtained by a process which comprises at least one step of recombinant DNA technology. Typically and preferably a preparation of a recombinant modified CuMV polypeptide comprises less than 10%, more preferably less than 5%, and still more preferably less than 1% recombinant modified CuMV polypeptide with an N-terminal methionine residue. Consequently, a recombinant virus-like particle of the invention may comprise otherwise identical recombinant polypeptides with and without an N-terminal methionine residue.

Recombinant virus-like particle: In the context of the invention the term “recombinant virus-like particle” refers to a virus-like particle (VLP) which is obtained by a process which comprises at least one step of recombinant DNA technology. Typically and preferably a recombinant VLP is obtained by expression of a recombinant viral coat protein in host, preferably in a bacterial cell. Typically and preferably, a recombinant virus-like particle comprises at least one recombinant polypeptide, preferably a recombinant CuMV polypeptide or recombinant modified CuMV polypeptide. Most preferably, a recombinant virus-like particle is composed of or consists of recombinant CuMV polypeptides or recombinant modified CuMV polypeptides. As a consequence, if in the context of the present invention the definition of inventive recombinant VLPs are effected with reference to specific amino acid sequences comprising a N-terminal methionine residue the scope of these inventive recombinant VLPs encompass the VLPs formed by said specific amino acid sequences without said N-terminal methionine residue but as well, even though typically in a minor amount as indicated herein, the VLPs formed by said specific amino acid sequences with said N-terminal methionine. Furthermore, it is within the scope of the present invention that if the definition of inventive recombinant VLPs are effected with reference to specific amino acid sequences comprising a N-terminal methionine residue VLPs are encompassed comprising both amino acid sequences comprising still said N-terminal methionine residue and amino acid sequences lacking the N-terminal methionine residue.

Mutated amino acid sequence: The term “mutated amino acid sequence” refers to an amino acid sequence which is obtained by introducing a defined set of mutations into an amino acid sequence to be mutated. In the context of the invention, said amino acid sequence to be mutated typically and preferably is an amino acid sequence of a coat protein of CuMV. Thus, a mutated amino acid sequence differs from an amino acid sequence of a coat protein of CuMV in at least one amino acid residue, wherein said mutated amino acid sequence and said amino acid sequence to be mutated show a sequence identity of at least 90%. Typically and preferably said mutated amino acid sequence and said amino acid sequence to be mutated show a sequence identity of at least 91%, 92%, 93% 94%, 95%, 96%, 97%, 98%, or 99%. Preferably, said mutated amino acid sequence and said sequence to be mutated differ in at most 11, 10, 9, 8, 7, 6, 4, 3, 2, or 1 amino acid residues, wherein further preferably said difference is selected from insertion, deletion and amino acid exchange. Preferably, the mutated amino acid sequence differs from an amino acid sequence of a coat protein of CuMV in least one amino acid, wherein preferably said difference is an amino acid exchange.

The position on an amino acid sequence, which is corresponding to given residues of another amino acid sequence can be identified by sequence alignment, typically and preferably by using the BLASTP algorithm, most preferably using the standard settings. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Sequence identity: The sequence identity of two given amino acid sequences is determined based on an alignment of both sequences. Algorithms for the determination of sequence identity are available to the artisan. Preferably, the sequence identity of two amino acid sequences is determined using publicly available computer homology programs such as the “BLAST” program (http://blast.ncbi.nlm.nih.gov/Blast.cgi) or the “CLUSTALW” (http://www.genome.jp/tools/clustalw/), and hereby preferably by the “BLAST” program provided on the NCBI homepage at http://blast.ncbi.nlm.nih.gov/Blast.cgi, using the default settings provided therein. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Amino acid exchange: The term amino acid exchange refers to the exchange of a given amino acid residue in an amino acid sequence by any other amino acid residue having a different chemical structure, preferably by another proteinogenic amino acid residue. Thus, in contrast to insertion or deletion of an amino acid, the amino acid exchange does not change the total number of amino acids of said amino acid sequence. Very preferred in the context of the invention is the exchange of an amino acid residue of said amino acid sequence to be mutated by a lysine residue or by a cysteine residue.

Epitope: The term epitope refers to continuous or discontinuous portions of an antigen, preferably a polypeptide, wherein said portions can be specifically bound by an antibody or by a T-cell receptor within the context of an MHC molecule. With respect to antibodies, specific binding excludes non-specific binding but does not necessarily exclude cross-reactivity. An epitope typically comprise 5-20 amino acids in a spatial conformation which is unique to the antigenic site.

T helper (Th) cell epitope: The term “T helper (Th) cell epitope” as used herein refers to an epitope that is capable of recognition by a helper Th cell. In another preferred embodiment, said T helper cell epitope is a universal T helper cell epitope. Universal Th cell epitope: The term “universal Th cell epitope” as used herein refers to a Th cell epitope that is capable of binding to at least one, preferably more than one MHC class II molecules. The simplest way to determine whether a peptide sequence is a universal Th cell epitope is to measure the ability of the peptide to bind to individual MHC class II molecules. This may be measured by the ability of the peptide to compete with the binding of a known Th cell epitope peptide to the MHC class II molecule. A representative selection of HLA-DR molecules are described in e.g. Alexander J, et al., Immunity (1994) 1:751-761. Affinities of Th cell epitopes for MHC class II molecules should be at least 10⁻⁵M. An alternative, more tedious but also more relevant way to determine the “universality” of a Th cell epitope is the demonstration that a larger fraction of people (>30%) generate a measurable T cell response upon immunization and boosting one months later with a protein containing the Th cell epitope formulated in IFA. A representative collection of MHC class II molecules present in different individuals is given in Panina-Bordignon P, et al., Eur J Immunol (1989) 19:2237-2242. As a consequence, the term “universal Th cell epitope” as used herein preferably refers to a Th cell epitope that generates a measurable T cell response upon immunization and boosting (one months later with a protein containing the Th cell epitope formulated in IFA) in more than 30% of a selected group of individuals as described in Panina-Bordignon P, et al., Eur J Immunol (1989) 19:2237-2242. Moreover, and again further preferred, the term “universal Th cell epitope” as used herein preferably refers to a Th cell epitope that is capable of binding to at least one, preferably to at least two, and even more preferably to at least three DR alleles selected from of DR1, DR2w2b, DR3, DR4w4, DR4w14, DR5, DR7, DR52a, DRw53, DR2w2a; and preferably selected from DR1, DR2w2b, DR4w4, DR4w14, DR5, DR7, DRw53, DR2w2a, with an affinity at least 500 nM (as described in Alexander J, et al., Immunity (1994) 1:751-761 and references cited herein); a preferred binding assay to evaluate said affinities is the one described by Sette A, et al., J Immunol (1989) 142:35-40. In an even again more preferable manner, the term “universal Th cell epitope” as used herein refers to a Th cell epitope that is capable of binding to at least one, preferably to at least two, and even more preferably to at least three DR alleles selected from DR1, DR2w2b, DR4w4, DR4w14, DR5, DR7, DRw53, DR2w2a, with an affinity at least 500 nM (as described in Alexander J, et al., Immunity (1994) 1:751-761 and references cited herein); a preferred binding assay to evaluate said affinities is the one described by Sette A, et al., J Immunol (1989) 142:35-40.

Universal Th cell epitopes are described, and known to the skilled person in the art, such as by Alexander J, et al., Immunity (1994) 1:751-761, Panina-Bordignon P, et al., Eur J Immunol (1989) 19:2237-2242, Calvo-Calle J M, et al., J Immunol (1997) 159:1362-1373, and Valmori D, et al., J Immunol (1992) 149:717-721.

Effective amount: As used herein, the term “effective amount” refers to an amount necessary or sufficient to realize a desired biologic effect. An effective amount of the composition, or alternatively the pharmaceutical composition, would be the amount that achieves this selected result, and such an amount could be determined as a matter of routine by a person skilled in the art. The effective amount can vary depending on the particular composition being administered and the size of the subject. One of ordinary skill in the art can empirically determine the effective amount of a particular composition of the present invention without necessitating undue experimentation.

Treatment: As used herein, the terms “treatment”, “treat”, “treated” or “treating” refer to prophylaxis and/or therapy. In one embodiment, the terms “treatment”, “treat”, “treated” or “treating” refer to a therapeutic treatment. In another embodiment, the terms “treatment”, “treat”, “treated” or “treating” refer to a prophylactic treatment.

Immunostimulatory substance: As used herein, the term “immunostimulatory substance” refers to a substance capable of inducing and/or enhancing an immune response. Immunostimulatory substances, as used herein, include, but are not limited to, toll-like receptor activating substances and substances inducing cytokine secretion. Toll-like receptor activating substances include, but are not limited to, immunostimulatory nucleic acids.

Immunostimulatory nucleic acid: As used herein, the term immunostimulatory nucleic acid refers to a nucleic acid capable of inducing and/or enhancing an immune response. Immunostimulatory nucleic acids comprise ribonucleic acids and desoxyribonucleic acids, wherein both, ribonucleic acids and desoxyribonucleic acids may be either double stranded or single stranded. Preferred ISS-NA are ribonucleic acids, wherein further preferably said ribonucleic acids are single stranded.

Packaged: The term “packaged” as used herein refers to the state of an immunostimulatory substances in relation to the VLP. The term “packaged” as used herein includes binding that may be covalent, e.g., by chemically coupling, or non-covalent, e.g., ionic interactions, hydrophobic interactions, hydrogen bonds, etc. The term also includes the enclosement, or partial enclosement, of an immunostimulatory substance. Thus, the immunostimulatory substances can be enclosed by the VLP without the existence of an actual binding, in particular of a covalent binding. In preferred embodiments, the at least one immunostimulatory substances is packaged inside the VLP, most preferably in a non-covalent manner. In case said immunostimulatory substances is nucleic acid, preferably a RNA, the term packaged implies that said nucleic acid is not accessible to nucleases hydrolysis, preferably not accessible to RNAse hydrolysis.

Several aspects of the present invention are disclosed herein; the embodiments and preferred embodiments, respectively, mentioned further herein are applicable for each and any aspect of the present invention disclosed herein, even though not explicitly mentioned.

In a first aspect, the present invention provides for a composition comprising particles, wherein each particle comprises, preferably consists of,
(a) at least one virus-like particle (VLP), preferably a recombinant VLP, and
(b) at least one microcrystalline tyrosine, preferably microcrystalline L-tyrosine, wherein said microcrystalline tyrosine has a median particle size of and between 0.1 μm and 50 μm, preferably of and between 1 μm and 10 μm, as preferably determined by Flow Particle Image Analyser (FPIA) and further preferably as described in Example 2; and
wherein said VLP does not comprise a tumor-associated antigen or a tumor-specific antigen associated with or covalently linked to said VLP, and wherein further preferably said VLP does not comprise an antigen (such as an allergen) associated with or covalently linked to said VLP.
Preferably said composition is capable of treating tumors, preferably malignant tumors, and further preferably is capable of treating solid tumors, preferably malignant solid tumors, and again further preferably said composition is capable of intratumorally treating malignant solid tumors.

In a further aspect, the present invention provides for a composition comprising, preferably consisting of,
(a) at least one virus-like particle (VLP), preferably a recombinant VLP, and
(b) at least one microcrystalline tyrosine, preferably microcrystalline L-tyrosine, wherein said microcrystalline tyrosine has a median particle size of and between 0.1 μm and 50 μm, preferably of and between 1 μm and 10 μm, as preferably determined by Flow Particle Image Analyser (FPIA) and further preferably as described in Example 2; and
wherein said VLP does not comprise a tumor-associated antigen or a tumor-specific antigen associated with or covalently linked to said VLP, and wherein further preferably said VLP does not comprise an antigen (such as an allergen) associated with or covalently linked to said VLP.
Preferably said composition is capable of treating tumors, preferably malignant tumors, and further preferably is capable of treating solid tumors, preferably malignant solid tumors, and again further preferably said composition is capable of intratumorally treating malignant solid tumors.

In again further aspect, the present invention provides for a composition comprising particles, wherein each particle comprises, preferably consists of,
(a) at least one virus-like particle (VLP), preferably a recombinant VLP, and
(b) at least one microcrystalline tyrosine, preferably microcrystalline L-tyrosine, wherein said microcrystalline tyrosine has a median particle size of and between 0.1 μm and 50 μm, preferably of and between 1 μm and 10 μm, as preferably determined by Flow Particle Image Analyser (FPIA) and further preferably as described in Example 2; and
wherein said VLP does not comprise a tumor-associated antigen associated with or covalently linked to said VLP, and wherein further preferably said VLP does not comprise an antigen (such as an allergen) associated with or covalently linked to said VLP.
Preferably said composition is capable of treating tumors, preferably malignant tumors, and further preferably is capable of treating solid tumors, preferably malignant solid tumors, and again further preferably said composition is capable of intratumorally treating malignant solid tumors.

In again further aspect, the present invention provides for a composition comprising, preferably consisting of,
(a) at least one virus-like particle (VLP), preferably a recombinant VLP, and
(b) at least one microcrystalline tyrosine, preferably microcrystalline L-tyrosine, wherein said microcrystalline tyrosine has a median particle size of and between 0.1 μm and 50 μm, preferably of and between 1 μm and 10 μm, as preferably determined by Flow Particle Image Analyser (FPIA) and further preferably as described in Example 2; and
wherein said VLP does not comprise a tumor-associated antigen associated with or covalently linked to said VLP, and wherein further preferably said VLP does not comprise an antigen (such as an allergen) associated with or covalently linked to said VLP.
Preferably said composition is capable of treating tumors, preferably malignant tumors, and further preferably is capable of treating solid tumors, preferably malignant solid tumors, and again further preferably said composition is capable of intratumorally treating malignant solid tumors.

In a preferred embodiment, said microcrystalline tyrosine has a median particle size of and between 0.2 μm and 30 μm, and wherein further preferably of and between 0.5 μm and 25 μm, as preferably determined by Flow Particle Image Analyser (FPIA) and further preferably as described in Example 2. In a further preferred embodiment, said microcrystalline tyrosine has a median particle size of and between 0.75 μm and 20 μm, and further preferably of and between 1 μm and 10 μm, as preferably determined by Flow Particle Image Analyser (FPIA) and further preferably as described in Example 2. In a preferred embodiment, said microcrystalline tyrosine has a median particle size of and between 2 μm and 8 μm, and further preferably of and between 3 μm and 7 μm, as preferably determined by Flow Particle Image Analyser (FPIA) and further preferably as described in Example 2. In a preferred embodiment, said microcrystalline tyrosine has a median particle size of and between 3.5 μm and 6 μm, and further preferably of and between 3.5 μm and 5.5 μm as preferably determined by Flow Particle Image Analyser (FPIA) and further preferably as described in Example 2.

In a preferred embodiment, said microcrystalline tyrosine has a 10^{th} percentile of its particle size of and between 0.01 μm and 5 μm, and wherein further preferably of and between 0.02 μm and 3 μm, and wherein again further preferably of and between 0.05 μm and 2.5 μm, as preferably determined by Flow Particle Image Analyser (FPIA) and further preferably as described in Example 2. In a further preferred embodiment, said microcrystalline tyrosine has a 10^{th} percentile of its particle size of and between 0.075 μm and 2 μm, and further preferably of and between 0.1 μm and 1 μm, as preferably determined by Flow Particle Image Analyser (FPIA) and further preferably as described in Example 2. In a preferred embodiment, said microcrystalline tyrosine has a 10^{th} percentile of its particle size of and between 0.2 μm and 0.9 μm, and further preferably of and between 0.3 μm and 0.8 μm, as preferably determined by Flow Particle Image Analyser (FPIA) and further preferably as described in Example 2. In a preferred embodiment, said microcrystalline tyrosine has a 10^{th} percentile of its particle size of and between 0.4 μm and 0.8 μm, and further preferably of and between 0.5 μm and 0.75 μm as preferably determined by Flow Particle Image Analyser (FPIA) and further preferably as described in Example 2.

In a preferred embodiment, said microcrystalline tyrosine has a 90^{th} percentile of its particle size of and between 0.5 μm and 250 μm, and wherein further preferably of and between 1 μm and 150 μm, and wherein again further preferably of and between 2.5 μm and 125 μm, as preferably determined by Flow Particle Image Analyser (FPIA) and further preferably as described in Example 2. In a further preferred embodiment, said microcrystalline tyrosine has a 90^{th} percentile of its particle size of and between 3.75 μm and 100 μm, and further preferably of and between 5 μm and 50 μm, as preferably determined by Flow Particle Image Analyser (FPIA) and further preferably as described in Example 2. In a preferred embodiment, said microcrystalline tyrosine has a 90^{th} percentile of its particle size of and between 10 μm and 40 μm, and further preferably of and between 10 μm and 35 μm, and wherein again further preferably of and between 10 μm and 30 μm, as preferably determined by Flow Particle Image Analyser (FPIA) and further preferably as described in Example 2. In a preferred embodiment, said microcrystalline tyrosine has a 90^{th} percentile of its particle size of and between 15 μm and 30 μm, and further preferably of and between 17.5 μm and 27.5 μm as preferably determined by Flow Particle Image Analyser (FPIA) and further preferably as described in Example 2.

In a preferred embodiment, said microcrystalline tyrosine has a maximum particle size of 750 μm, and wherein further preferably of 500 μm, and wherein again further preferably of 400 μm, and wherein again further preferably of 300 μm, as preferably determined by Flow Particle Image Analyser (FPIA) and further preferably as described in Example 2. In a further preferred embodiment, said microcrystalline tyrosine has maximum particle size of 250 μm, and further preferably of 225 μm, as preferably determined by Flow Particle Image Analyser (FPIA) and further preferably as described in Example 2. In a preferred embodiment, said microcrystalline tyrosine has maximum particle size of 200 μm, and further preferably of 175 μm, and wherein again further preferably of 150 μm, as preferably determined by Flow Particle Image Analyser (FPIA) and further preferably as described in Example 2. In a preferred embodiment, said microcrystalline tyrosine maximum particle size of 125 μm, and further preferably of 100 μm as preferably determined by Flow Particle Image Analyser (FPIA) and further preferably as described in Example 2.

In a further preferred embodiment, said microcrystalline tyrosine has a maximum particle size of 750 μm, wherein preferably said microcrystalline tyrosine has a maximum particle size of 150 μm.

In a further preferred embodiment, said VLP is adsorbed on said microcrystalline tyrosine.

In a preferred embodiment, said composition is an aqueous composition, wherein preferably said composition comprises a buffer solution, further preferably a phosphate buffered saline. In a preferred embodiment, said composition is an aqueous composition, wherein preferably said composition comprises a phosphate buffered saline. In a preferred embodiment, said composition is an aqueous composition comprising a phosphate buffered saline.

In a further preferred embodiment, said composition is an aqueous composition, wherein preferably the concentration of said microcrystalline tyrosine, preferably said microcrystalline L-tyrosine, is 0.5% to 10% (weight tyrosine/volume solution), and wherein preferably the concentration of said microcrystalline tyrosine, preferably said microcrystalline L-tyrosine, is 1% to 7.5% (weight tyrosine/volume solution).

In a preferred embodiment, said composition is an aqueous composition, wherein the concentration of said microcrystalline tyrosine, preferably said microcrystalline L-tyrosine, is 0.5% to 10% (weight tyrosine/volume solution), preferably 1% to 7.5%, further preferably 2% to 7%. In another preferred embodiment, said composition is an aqueous composition, wherein the concentration of said microcrystalline tyrosine, is 2.5% to 6% (weight tyrosine/volume solution), preferably 3% to 5%. In another preferred embodiment, the concentration of said microcrystalline tyrosine is 3.5% to 4.5% (weight tyrosine/volume solution), further of about 4% (weight tyrosine/volume solution). In another preferred embodiment, the concentration of said microcrystalline tyrosine is of about 1% (weight tyrosine/volume solution). In another preferred embodiment, the concentration of said microcrystalline tyrosine is of about 1.5% (weight tyrosine/volume solution). In another preferred embodiment, the concentration of said microcrystalline tyrosine is of about 2% (weight tyrosine/volume solution). In another preferred embodiment, the concentration of said microcrystalline tyrosine is of about 2.5% (weight tyrosine/volume solution). In another preferred embodiment, the concentration of said microcrystalline tyrosine is of about 3% (weight tyrosine/volume solution). In another preferred embodiment, the concentration of said microcrystalline tyrosine is of about 3.5% (weight tyrosine/volume solution). In another preferred embodiment, the concentration of said microcrystalline tyrosine is of about 4% (weight tyrosine/volume solution). In another preferred embodiment, the concentration of said microcrystalline tyrosine is of about 4.5% (weight tyrosine/volume solution). In another preferred embodiment, the concentration of said microcrystalline tyrosine is of about 5% (weight tyrosine/volume solution). In another preferred embodiment, the concentration of said microcrystalline tyrosine is of about 5.5% (weight tyrosine/volume solution). In another preferred embodiment, the concentration of said microcrystalline tyrosine is of about 6% (weight tyrosine/volume solution).

In a further preferred embodiment, said microcrystalline tyrosine is obtained by a method comprises the steps of
(i) providing an aqueous NaOH solution, wherein said aqueous NaOH solution has a molar concentration of 1M to 5M, preferably of 3M-4M, more preferably 3.2M to 3.8M, further preferably 3.2M or 3.8M;
(ii) providing an aqueous HCl solution, wherein said aqueous HCl solution has a molar concentration of 1M to 5M, preferably of 3M-4M, more preferably 3.8M;
(iii) dissolving tyrosine, preferably L-tyrosine, in said aqueous HCl solution leading to a HCl solution comprising tyrosine, wherein the concentration of said tyrosine in said HCl solution is between 5-40% (weight tyrosine/volume HCl solution), preferably between 10-30% (weight tyrosine/volume HCl solution), more preferably 24% (weight tyrosine/volume HCl solution);
(iv) co-precipitating said microcrystalline tyrosine by combining and homogenizing, preferably equal volume amounts of, said aqueous NaOH solution and said HCl solution comprising tyrosine, wherein preferably said co-precipitating further comprises the addition of Evans solution in the presence of a phosphate buffer;
(v) separating said microcrystalline tyrosine and optionally purifying said microcrystalline tyrosine; and
(vi) optionally suspending said microcrystalline tyrosine in a buffer solution, preferably in phosphate buffered saline, to a concentration of 2% to 5% (weight tyrosine/volume solution), preferably to a concentration of 3.5% to 4.5% (weight tyrosine/volume solution), further preferably to a concentration of 4% (weight tyrosine/volume solution).

In a preferred embodiment, said aqueous HCl solution comprising tyrosine and said aqueous NaOH solution are sterile filtered prior to said co-precipitating. In a preferred embodiment, said molar concentrations of said aqueous HCl solution and said aqueous NaOH solution, and said volume amounts used in the co-precipitating are selected such that the final pH of the microcrystalline tyrosine suspension is between 5.0 and 7.0.

In a preferred embodiment, said VLP is derived from a plant virus or is a VLP of an RNA bacteriophage.

In a preferred embodiment, said VLP is derived from a plant virus. In a preferred embodiment, said VLP is a VLP of an RNA bacteriophage. In a preferred embodiment, said VLP is derived from a plant virus selected from the group consisting of cucumber mosaic virus (CuMV), cowpea chlorotic mottle virus (CCMV) and tobacco mosaic virus (TMV). In a preferred embodiment, said VLP is derived from cucumber mosaic virus (CuMV). In a preferred embodiment, said VLP is derived from cowpea chlorotic mottle virus (CCMV). In a preferred embodiment, said VLP is a VLP of an RNA bacteriophage. In a preferred embodiment, said VLP is a VLP of an RNA bacteriophage, wherein said RNA bacteriophage is selected from the group consisting of Qbeta and AP205. In a preferred embodiment, said VLP is a VLP of an RNA bacteriophage Qbeta. In a preferred embodiment, said VLP is a VLP of an RNA bacteriophage AP205.

In a preferred embodiment, said VLP is derived from a plant virus or is a VLP of an RNA bacteriophage, wherein preferably said plant virus is selected from the group consisting of cucumber mosaic virus (CuMV), cowpea chlorotic mottle virus (CCMV) and tobacco mosaic virus (TMV), and wherein preferably said VLP of an RNA bacteriophage is a VLP of an RNA bacteriophage Qbeta or a VLP of an RNA bacteriophage AP205.

In a further preferred embodiment, said VLP comprises, consists essentially of, or alternatively consists of, recombinant coat proteins of an RNA bacteriophage, and wherein preferably said VLP comprises, preferably consists of, recombinant coat proteins of RNA bacteriophage Qβ or of RNA bacteriophage AP205, and wherein further preferably said VLP comprises, preferably consists of, recombinant coat proteins of RNA bacteriophage Qβ. In a further preferred embodiment, said VLP comprises, preferably consists of, recombinant coat proteins comprising or preferably consisting of an amino acid sequence selected from (a) SEQ ID NO:14; (b) a mixture of SEQ ID NO:14 and SEQ ID NO:15; or (c) SEQ ID NO:16. In a further preferred embodiment, said VLP is a VLP of RNA bacteriophage Qβ. In a further preferred embodiment, said VLP comprises, consists essentially of, or alternatively consists of, recombinant coat proteins of RNA bacteriophage Qβ. Again in a further preferred embodiment, said VLP comprises, consists essentially of, or alternatively consists of, recombinant coat proteins comprising or preferably consisting of SEQ ID NO:14.

In another preferred embodiment, said core particle is a virus-like particle (VLP) wherein said VLP is a VLP of RNA bacteriophage Qβ, and said VLP comprises, consists essentially of, or alternatively consists of, recombinant coat proteins of RNA bacteriophage Qβ, and wherein said recombinant coat proteins comprising or preferably consisting of SEQ ID NO:14.

In a preferred embodiment, said VLP is a VLP of cucumber mosaic virus (CuMV), wherein preferably said VLP of cucumber mosaic virus (CuMV) comprises, preferably consists of, at least one recombinant coat protein or a mutated amino acid sequence, wherein said mutated amino acid sequence and said coat protein of CuMV show a sequence identity of at least 90%, preferably of at least 95%, further preferably of at least 98% and again more preferably of at least 99%.

In a further preferred embodiment, said VLP is a VLP of cucumber mosaic virus (CuMV), wherein preferably said VLP of cucumber mosaic virus (CuMV) comprises, preferably consists of, at least one recombinant coat protein of SEQ ID NO:1, or a mutated amino acid sequence, wherein said mutated amino acid sequence and said coat protein of CuMV of SEQ ID NO:1 show a sequence identity of at least 90%, preferably of at least 95%, further preferably of at least 98% and again more preferably of at least 99%.

In a further preferred embodiment, said VLP is a modified VLP, wherein said modified VLP comprises, preferably consists of, at least one modified VLP polypeptide, wherein said modified VLP polypeptide comprises,
(a) a VLP polypeptide, and
(b) a T helper cell epitope,
wherein said VLP polypeptide comprises, or preferably consists of,
(i) an amino acid sequence of a coat protein of a virus, preferably an amino acid sequence of a coat protein of a plant virus; or
(ii) a mutated amino acid sequence, wherein said mutated amino acid sequence and said coat protein of a virus show a sequence identity of at least 90%, preferably of at least 95%, further preferably of at least 98% and again more preferably of at least 99%.

In a further preferred embodiment, said VLP is a modified VLP of cucumber mosaic virus (CuMV), wherein said modified VLP of CuMV comprises, preferably consists of, at least one modified CuMV polypeptide, wherein said modified CuMV polypeptide comprises, or preferably consists of,
(a) a CuMV polypeptide, and
(b) a T helper cell epitope; and
   wherein said CuMV polypeptide comprises, or preferably consists of,
   (i) an amino acid sequence of a coat protein of CuMV; or
   (ii) a mutated amino acid sequence, wherein said mutated amino acid sequence and said coat protein of CuMV show a sequence identity of at least 90%, preferably of at least 95%, further preferably of at least 98% and again more preferably of at least 99%.

In a preferred embodiment, said CuMV polypeptide is a coat protein of CuMV or an amino acid sequence having a sequence identity of at least 75%, preferably 85% with SEQ ID NO:1. In a preferred embodiment, said CuMV polypeptide is a coat protein of CuMV or an amino acid sequence having a sequence identity of at least 90%, preferably 95% with SEQ ID NO:1. In a preferred embodiment, said CuMV polypeptide is a coat protein of CuMV with SEQ ID NO: 1. In a preferred embodiment, said coat protein of CuMV comprises SEQ ID NO:1. In a preferred embodiment, said coat protein of CuMV consists of SEQ ID NO:1. In a preferred embodiment, said CuMV polypeptide comprises a coat protein of CuMV. In a preferred embodiment, said CuMV polypeptide consists of a coat protein of CuMV. In a preferred embodiment, said CuMV polypeptide comprises a coat protein of CuMV, wherein said coat protein of CuMV comprises SEQ ID NO:1. In a preferred embodiment, said CuMV polypeptide comprises a coat protein of CuMV, wherein said coat protein of CuMV consists of SEQ ID NO:1. In a preferred embodiment, said CuMV polypeptide consists of a coat protein of CuMV, wherein said coat protein of CuMV consists of SEQ ID NO:1.

In a preferred embodiment, said CuMV polypeptide comprises SEQ ID NO:2 or an amino acid sequence region, wherein said amino acid sequence region has a sequence identity of at least 75% with SEQ ID NO:2. In a preferred embodiment, said CuMV polypeptide comprises SEQ ID NO:2 or an amino acid sequence region, wherein said amino acid sequence region has a sequence identity of at least 80% with SEQ ID NO:2. In a preferred embodiment, said CuMV polypeptide comprises SEQ ID NO:2 or an amino acid sequence region, wherein said amino acid sequence region has a sequence identity of at least 85% with SEQ ID NO:2. In a preferred embodiment, said CuMV polypeptide comprises SEQ ID NO:2 or an amino acid sequence region, wherein said amino acid sequence region has a sequence identity of at least 90% with SEQ ID NO:2. In a preferred embodiment, said CuMV polypeptide comprises SEQ ID NO:2 or an amino acid sequence region, wherein said amino acid sequence region has a sequence identity of at least 95% with SEQ ID NO:2. In a preferred embodiment, said CuMV polypeptide comprises SEQ ID NO:2 or an amino acid sequence region, wherein said amino acid sequence region has a sequence identity of at least 98% with SEQ ID NO:2. In a preferred embodiment, said CuMV polypeptide comprises SEQ ID NO:2 or an amino acid sequence region, wherein said amino acid sequence region has a sequence identity of at least 99% with SEQ ID NO:2.

In a preferred embodiment, said CuMV polypeptide comprises, or preferably consists of, (i) an amino acid sequence of a coat protein of CuMV, wherein said amino acid sequence comprises, or preferably consists of, SEQ ID NO: 1; or (ii) an amino acid sequence having a sequence identity of at least 90% of SEQ ID NO:1; and wherein said amino sequence as defined in (i) or (ii) comprises SEQ ID NO:2 or an amino acid sequence region, wherein said amino acid sequence region has a sequence identity of at least 90% with SEQ ID NO:2. In a preferred embodiment, said CuMV polypeptide comprises, or preferably consists of, (i) an amino acid sequence of a coat protein of CuMV, wherein said amino acid sequence comprises, or preferably consists of, SEQ ID NO:1; or (ii) an amino acid sequence having a sequence identity of at least 95% of SEQ ID NO:1; and wherein said amino sequence as defined in (i) or (ii) comprises SEQ ID NO:2 or an amino acid sequence region, wherein said amino acid sequence region has a sequence identity of at least 95% with SEQ ID NO:2. In a preferred embodiment, said CuMV polypeptide comprises, or preferably consists of, (i) an amino acid sequence of a coat protein of CuMV, wherein said amino acid sequence comprises, or preferably consists of, SEQ ID NO:1; or (ii) an amino acid sequence having a sequence identity of at least 90% of SEQ ID NO: 1; and wherein said amino sequence as defined in (i) or (ii) comprises SEQ ID NO:2.

In a preferred embodiment, said modified CuMV polypeptide further comprises a T helper cell epitope, wherein said T helper cell epitope replaces a N-terminal region of said CuMV polypeptide.

In a preferred embodiment, the number of amino acids of said N-terminal region replaced is equal to or lower than the number of amino acids of which said T helper cell epitope consists.

In a preferred embodiment, said replaced N-terminal region of said CuMV polypeptide consists of 5 to 15 consecutive amino acids. In a preferred embodiment, said replaced N-terminal region of said CuMV polypeptide consists of 9 to 14 consecutive amino acids. In a preferred embodiment, said replaced N-terminal region of said CuMV polypeptide consists of 11 to 13 consecutive amino acids. In a preferred embodiment, said N-terminal region of said CuMV polypeptide corresponds to amino acids 2-12 of SEQ ID NO:1. In a preferred embodiment, said modified CuMV polypeptide further comprises a T helper cell epitope, wherein said T helper cell epitope replaces a N-terminal region of said CuMV polypeptide, and wherein said N-terminal region of said CuMV polypeptide corresponds to amino acids 2-12 of SEQ ID NO:1. In a preferred embodiment, said T helper cell epitope is a universal T helper cell epitope. In a preferred embodiment, said T helper cell epitope consists of at most 20 amino acids.

In a preferred embodiment, the Th cell epitope is selected from TT 830-843 (SEQ ID NO:3), PADRE (SEQ ID NO:4), TT 947-967 (SEQ ID NO:5), HA 307-319 (SEQ ID NO:6), HBVnc 50-69 (SEQ ID NO:7), CS 378-398 (SEQ ID NO:8) and MT 17-31 (SEQ ID NO:9). In a very preferred embodiment, said Th cell epitope is a Th cell epitope derived from tetanus toxin or is a PADRE sequence. In a preferred embodiment, said T helper cell epitope is derived from a human vaccine. In a very preferred embodiment, said Th cell epitope is a Th cell epitope derived from tetanus toxin. In a preferred embodiment, said Th cell epitope is a PADRE sequence. In a very preferred embodiment, said Th cell epitope comprises the amino acid sequence of SEQ ID NO:3 or SEQ ID NO:4. In a very preferred embodiment, said Th cell epitope consists of the amino acid sequence of SEQ ID NO:3 or SEQ ID NO:4. In a very preferred embodiment, said Th cell epitope comprises the amino acid sequence of SEQ ID NO:3. In a preferred embodiment, said Th cell epitope consists of the amino acid sequence of SEQ ID NO:3. In a very preferred embodiment, said Th cell epitope comprises the amino acid sequence of SEQ ID NO:4. In a very preferred embodiment, said Th cell epitope consists of the amino acid sequence of SEQ ID NO:4.

In a preferred embodiment, said CuMV polypeptide comprises, or preferably consists of, an amino acid sequence of a coat protein of CuMV, wherein said amino acid sequence comprises, or preferably consists of, SEQ ID NO:1 or an amino acid sequence having a sequence identity of at least 95% of SEQ ID NO:1; and wherein said amino sequence comprises SEQ ID NO:2, and wherein said T helper cell epitope replaces the N-terminal region of said CuMV polypeptide, and wherein said replaced N-terminal region of said CuMV polypeptide consists of 11 to 13 consecutive amino acids, preferably of 11 consecutive amino acids, and wherein further preferably said N-terminal region of said CuMV polypeptide corresponds to amino acids 2-12 of SEQ ID NO:1.

In another very preferred embodiment, said modified CMV polypeptide comprises the amino acid sequence of SEQ ID NO: 11. In another very preferred embodiment, said modified CMV polypeptide consists of the amino acid sequence of SEQ ID NO:11. In another very preferred embodiment, said modified CMV polypeptide comprises the amino acid sequence of SEQ ID NO:12. In another very preferred embodiment, said modified CMV polypeptide consists of the amino acid sequence of SEQ ID NO: 12.

In a further very preferred embodiment, the present invention provides for a composition comprising particles, wherein each particle comprises, preferably consists of,
(a) at least one virus-like particle (VLP), preferably a recombinant VLP, wherein said VLP is a modified VLP of cucumber mosaic virus (CuMV), wherein said modified VLP of CuMV comprises, preferably consists of, at least one modified CuMV polypeptide, wherein said modified CuMV polypeptide comprises, or preferably consists of, the amino acid sequence of SEQ ID NO:11;
(b) at least one microcrystalline tyrosine, preferably microcrystalline L-tyrosine, wherein said microcrystalline tyrosine has a median particle size of and between 0.1 μm and 50 μm, preferably of and between 1 μm and 10 μm, as preferably determined by Flow Particle Image Analyser (FPIA) and further preferably as described in Example 2; and
wherein said VLP does not comprise a tumor-associated antigen or a tumor-specific antigen covalently linked to said VLP, and wherein further preferably said VLP does not comprise an antigen covalently linked to said VLP.

In a further very preferred embodiment, the present invention provides for a composition comprising, preferably consisting of,
(a) at least one virus-like particle (VLP), preferably a recombinant VLP, wherein said VLP is a modified VLP of cucumber mosaic virus (CuMV), wherein said modified VLP of CuMV comprises, preferably consists of, at least one modified CuMV polypeptide, wherein said modified CuMV polypeptide comprises, or preferably consists of, the amino acid sequence of SEQ ID NO:11;
(b) at least one microcrystalline tyrosine, preferably microcrystalline L-tyrosine, wherein said microcrystalline tyrosine has a median particle size of and between 0.1 μm and 50 μm, preferably of and between 1 μm and 10 μm, as preferably determined by Flow Particle Image Analyser (FPIA) and further preferably as described in Example 2; and
wherein said VLP does not comprise a tumor-associated antigen or a tumor-specific antigen covalently linked to said VLP, and wherein further preferably said VLP does not comprise an antigen covalently linked to said VLP.

In a further very preferred embodiment, the present invention provides for a composition comprising particles, wherein each particle comprises, preferably consists of,
(a) at least one virus-like particle (VLP), preferably a recombinant VLP, wherein said VLP is a modified VLP of cucumber mosaic virus (CuMV), wherein said modified VLP of CuMV comprises, preferably consists of, at least one modified CuMV polypeptide, wherein said modified CuMV polypeptide comprises, or preferably consists of, the amino acid sequence of SEQ ID NO:11;
(b) at least one microcrystalline tyrosine, preferably microcrystalline L-tyrosine, wherein said microcrystalline tyrosine has a median particle size of and between 0.1 μm and 50 μm, preferably of and between 1 μm and 10 μm, as preferably determined by Flow Particle Image Analyser (FPIA) and further preferably as described in Example 2; and
wherein said VLP does not comprise a tumor-associated antigen covalently linked to said VLP, and wherein further preferably said VLP does not comprise an antigen covalently linked to said VLP.

In a further very preferred embodiment, the present invention provides for a composition comprising, preferably consisting of,
(a) at least one virus-like particle (VLP), preferably a recombinant VLP, wherein said VLP is a modified VLP of cucumber mosaic virus (CuMV), wherein said modified VLP of CuMV comprises, preferably consists of, at least one modified CuMV polypeptide, wherein said modified CuMV polypeptide comprises, or preferably consists of, the amino acid sequence of SEQ ID NO:11;
(b) at least one microcrystalline tyrosine, preferably microcrystalline L-tyrosine, wherein said microcrystalline tyrosine has a median particle size of and between 0.1 μm and 50 μm, preferably of and between 1 μm and 10 μm, as preferably determined by Flow Particle Image Analyser (FPIA) and further preferably as described in Example 2; and
wherein said VLP does not comprise a tumor-associated antigen covalently linked to said VLP, and wherein further preferably said VLP does not comprise an antigen covalently linked to said VLP.

In a further preferred embodiment, said composition does not comprise a specific cancer antigen. In a further preferred embodiment, said composition does not comprise a tumor-associated antigen. In a further preferred embodiment, said composition does not comprise a tumor-specific antigen.

In a further very preferred embodiment, said VLP does not comprise a specific cancer antigen covalently linked to said VLP, and wherein further preferably said VLP does not comprise an antigen covalently linked to said VLP. In a further very preferred embodiment, said VLP does not comprise a tumor-associated antigen covalently linked to said VLP, and wherein further preferably said VLP does not comprise an antigen covalently linked to said VLP. In a further very preferred embodiment, said VLP does not comprise a tumor-specific antigen covalently linked to said VLP, and wherein further preferably said VLP does not comprise an antigen covalently linked to said VLP. In a further very preferred embodiment, said VLP does not comprise the CTL epitope p33 derived from the lymphocytic choriomeningitis virus glycoprotein covalently linked to said VLP, and wherein further preferably said VLP does not comprise an antigen covalently linked to said VLP. In a further very preferred embodiment, said VLP, preferably said recombinant VLP, comprises an immunostimulatory substance packaged in said VLP, wherein preferably said immunostimulatory substance is a toll-like receptor activating substance. In a further very preferred embodiment, said toll-like receptor activating substance is a toll-like receptor 7 and/or 8 activating substance, and wherein preferably said toll-like receptor 7 and/or 8 activating substance is ssRNA. In a further very preferred embodiment, said toll-like receptor activating substance is an immunostimulatory nucleic acid, and wherein preferably said immunostimulatory nucleic acid is ssRNA. In a very preferred embodiment, said composition is capable of treating tumors, preferably malignant tumors, and wherein further preferably said composition is capable of treating solid tumors, preferably malignant solid tumors and again further preferably said composition is capable of intratumorally treating malignant solid tumors. In a further very preferred embodiment, said malignant solid tumors are selected from breast, prostate, colorectal, lung and melanoma. In a further very preferred embodiment, said malignant solid tumor is a breast malignant tumor. In a further very preferred embodiment, said malignant solid tumor is a prostate malignant tumor. In a further very preferred embodiment, said malignant solid tumor is a colorectal malignant tumor. In a further very preferred embodiment, said malignant solid tumor is a lung malignant tumor. In a further very preferred embodiment, said malignant solid tumor is a melanoma malignant tumor.

In a further aspect, the present invention provides for a pharmaceutical composition comprising the inventive composition and a pharmaceutically acceptable carrier.

In a further aspect, the present invention provides for a composition, as described and claimed herein, for use in a method of treating a solid tumor in a patient, wherein said patient is a human patient or an animal patient, and wherein preferably said animal patient is selected from an equine animal, preferably a horse, a dog, or a cat, and wherein further preferably said animal patient is a horse. In a very preferred embodiment, said method of treating a solid cancer in a patient is a method of intratumorally treating said solid cancer in a patient. In a further very preferred embodiment, said solid cancer is selected from breast cancer, prostate cancer, colorectal cancer, lung cancer and melanoma cancer. In a further very preferred embodiment, said solid cancer is breast cancer. In a further very preferred embodiment, said solid cancer is prostate cancer. In a further very preferred embodiment, said solid cancer is colorectal cancer. In a further very preferred embodiment, said solid cancer is lung cancer. In a further very preferred embodiment, said solid cancer is melanoma cancer. In a further very preferred embodiment, said method comprises administration of said inventive composition to said patient, wherein said administration is an intratumoral injection of said composition to said patient. In a further very preferred embodiment, said method comprises administration of said inventive composition to said patient, wherein said administration of said composition is into or substantially adjacent to the tumor of said patient.

In a further aspect, the present invention provides for the use of a composition, as described and claimed herein, for the manufacture of a medicament for treating a solid tumor in a patient, wherein said patient is a human patient or an animal patient, and wherein preferably said animal patient is selected from an equine animal, preferably a horse, a dog, or a cat, and wherein further preferably said animal patient is a horse. In a very preferred embodiment, said treating a solid cancer in a patient is intratumorally treating said solid cancer in a patient. In a further very preferred embodiment, said solid cancer is selected from breast cancer, prostate cancer, colorectal cancer, lung cancer and melanoma cancer. In a further very preferred embodiment, said solid cancer is breast cancer. In a further very preferred embodiment, said solid cancer is prostate cancer. In a further very preferred embodiment, said solid cancer is colorectal cancer. In a further very preferred embodiment, said solid cancer is lung cancer. In a further very preferred embodiment, said solid cancer is melanoma cancer. In a further very preferred embodiment, said treating a solid cancer in a patient is an intratumoral injection of said composition to said patient. In a further very preferred embodiment, said treating a solid cancer in a patient is administration of said composition into or substantially adjacent to the tumor of said patient.

In again a further aspect, the present invention provides for a method of treating a solid tumor in a patient, wherein said patient is a human patient or an animal patient, and wherein preferably said animal patient is selected from an equine animal, preferably a horse, a dog, or a cat, and wherein further preferably said animal patient is a horse, comprising administering to said human patient or animal patient an effective amount of a composition, as described and claimed herein, or a pharmaceutical composition. In a very preferred embodiment, said method of treating a solid cancer in a patient is a method of intratumorally treating said solid cancer in a patient. In a further very preferred embodiment, said solid cancer is selected from breast cancer, prostate cancer, colorectal cancer, lung cancer and melanoma cancer. In a further very preferred embodiment, said solid cancer is breast cancer. In a further very preferred embodiment, said solid cancer is prostate cancer. In a further very preferred embodiment, said solid cancer is colorectal cancer. In a further very preferred embodiment, said solid cancer is lung cancer. In a further very preferred embodiment, said solid cancer is melanoma cancer. In a further very preferred embodiment, said method comprises administration of said inventive composition to said patient, wherein said administration is an intratumoral injection of said composition to said patient. In a further very preferred embodiment, said method comprises administration of said inventive composition to said patient, wherein said administration of said composition is into or substantially adjacent to the tumor of said patient.

### EXAMPLES

Preferred virus-like particles (VLPs), in particular recombinant VLPs, as used herein have been prepared as described. Thus, in one embodiment, the VLP is VLP of RNA bacteriophage Qβ comprising, preferably consisting of, recombinant coat proteins of RNA bacteriophage Qβ of SEQ ID NO: 14. Such virus-like particles of RNA bacteriophages are disclosed in WO 02/056905, the disclosure of which is herewith incorporated by reference in its entirety. In particular Example 18 of WO 02/056905 contains a detailed description of the preparation of VLP particles of RNA bacteriophage Qβ. In a further embodiment, the VLP is a VLP of cowpea chlorotic mottle virus (CCMV), in particular, a modified VLP of CCMV, wherein a T helper cell epitope are incorporated at the N-terminus or at the C-terminus. In a further embodiment, the VLP is CCMVtt830 as described in Zinkhan S et al, Journal of Controlled Release (2021) 331:296-308, the disclosure of which is herewith incorporated by reference in its entirety. In a very preferred embodiment, the VLP is a VLP of cucumber mosaic virus (CuMV), in particular, a modified VLP of CuMV, wherein T helper cell epitopes replace N-terminal regions of the CuMV polypeptide. In a very preferred embodiment, the VLP is CuMVtt830 comprising modified CuMV polypeptides of SEQ ID NO:11 or CuMV-Npadr comprising modified CuMV polypeptides of SEQ ID NO:12, preferably CuMVtt830 comprising modified CuMV polypeptides of SEQ ID NO:11, as described herein and as disclosed in WO 2016/062720. It is of note that in WO 2016/062720 cucumber mosaic virus is abbreviated as CMV. In particular Examples 1 to 6 of WO 2016/062720 contain a detailed description of the preparation of VLP particles of modified CuMV polypeptides of SEQ ID NO:11 and SEQ ID NO:12.

### Example 1

### Preparation of Microcrystalline Tyrosine (MCT)

Microcrystalline tyrosine (MCT) was prepared similarly as described in Bell A J et al, 2015, Journal of Inorganic Biochemistry 152:147-153). In brief, microcrystalline tyrosine (MCT) is produced by the co-precipitation of 24% w/v L-tyrosine solution in 3.8M hydrochloric acid with 3.2M sodium hydroxide. The co-precipitation is completed in Evans solution in the presence of phosphate buffer. The product is washed with phosphate buffered saline across a stainless steel sintered filter to reduce excess chloride content. The product is collected as a microcrystalline suspension in phosphate buffered saline at a concentration of 4% w/v L-tyrosine.

The following two preparations have been prepared having the following particle sizes as measured by FPIA as described in Example 2.

| | Preparation 1 [μm] | Preparation 2 [μm] |
|---|---|---|
| 10^{th} Percentile* | 0.66 | 0.72 |
| 50^{th} Percentile | 4.31 | 4.01 |
| (median particle size)* | | |
| 90^{th} Percentile* | 22.25 | 22.12 |
| Maximum particle size* | 81.20 | 60.66 |

*values correspond to mean values calculated from three samples of the preparation.

### Example 2

Particle Size Analysis Using Malvern Flow Particle Image Analyser 3000 The standard and preferred analytical method for determining and analyzing particle size and particle distribution for the microcrystalline tyrosine, preferably the microcrystalline L-tyrosine in accordance with the present invention, is performed by using the Malvern Instruments FPIA 3000, wherein FPIA stands for Flow Particle Image Analyser (FPIA).

Traditional particle size methods such as laser diffraction can view a monodisperse sample as polydisperse due to random particle orientation. The FPIA instrument is an image analyser that utilises a ‘sheath flow’ principle to orientate and align particles so that a more uniform and accurate result of particle shape and size can be found. This is achieved by ensuring that the largest area of the particle is facing the image detector. The FPIA utilises a charge-coupled device (CCD) camera to capture pictures of particles as they pass in this controlled manner through a flow cell and analysed in ‘real-time’.

The FPIA is able to detect a range of 1.5 μm-160 μm by using two different lenses, one to give a quoted detection range of 1.5 μm-40 μm using the High Power Field (HPF) and a second to give a quoted detection range 8 μm-160 μm using the Low Power Field (LPF). A maximum of 36,000 particles can be photographed and measured in one run with a total analysis time of 2 minutes 30 seconds.

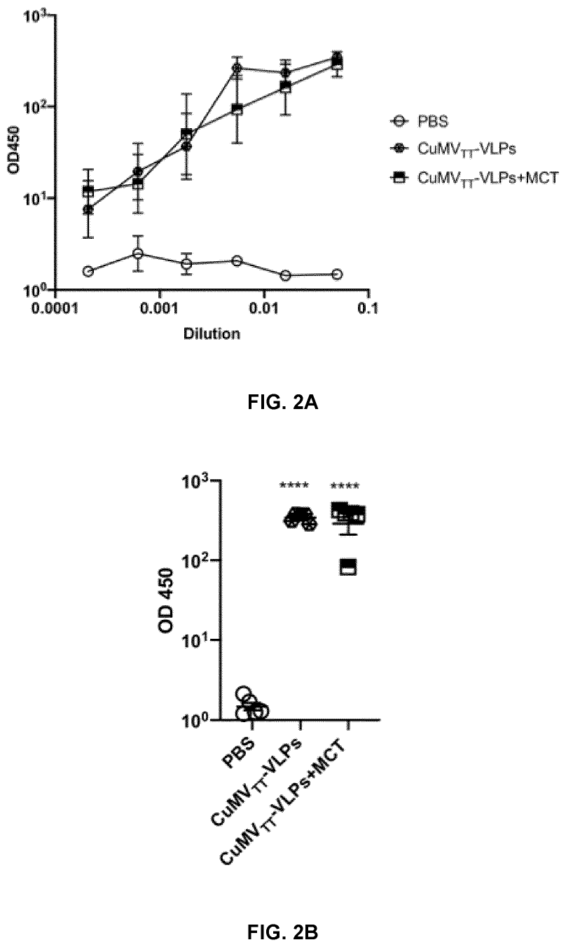

The particle size for the microcrystalline tyrosine, preferably the microcrystalline L-tyrosine, as used and described herein, refers to the Equivalent Circular Perimeter Diameter Dₚ as described and defined in Li et al (Li et al., Particulate Science and Technology, 23: 265-284, 2005; it is in particular referred to FIG. 2(b) and equation (2) on page 271). The median particle size, as used herein, refers to the 50^{th} percentile corresponding to the particle size at or below which 50% of all the measured particle sizes in the distribution and composition, respectively, are determined as Dₚ by FPIA, typically and preferably, as described in this Example. Further values, typically and preferably, measured by FPIA for the microcrystalline tyrosine, preferably the microcrystalline L-tyrosine are the 10^{th} percentile, the 90^{th} percentile and the maximum particle size. The 10^{th} percentile refers to the size at or below which 10% of all the measured particle sizes in the distribution and composition, respectively, are determined as Dₚ by FPIA, typically and preferably, as described in this Example. The 90^{th} percentile refers to the size at or below which 90% of all the measured particle sizes in the distribution and composition, respectively, are determined as Dₚ by FPIA, typically and preferably, as described in this Example, while the maximum particle size refers to the highest particle size value measured within the distribution and composition, respectively.

### Example 3

### Preparation of a Composition Comprising CuMVtt830-VLP and MCT

A composition of CuMVtt830-VLP with 4% w/v MCT was prepared by mixing both ingredients in a shaker at room temperature for 1 hour allowing the nano-sized VLPs to decorate the micron-sized MCT crystals, analogously as described in Mohsen MO et al (Journal for ImmunoTherapy of Cancer (2020) 7(1):114). Qβ-VLPs formulated with 4% w/v MCT or CuMV_{TT}-VLPs formulated with 4% w/v MCT were prepared accordingly.

### Example 4

### Anti-Tumor Efficacy of the Composition Comprising CuMVtt830-VLP and MCT

The composition comprising CuMVtt830-VLP with MCT was tested in an aggressive transplanted murine melanoma model which is illustrated in FIG. 1A, and described as follows: 1×10⁶ cells of B16F10 melanoma cell line were injected into the flank of RAG2^{-/-} C57/BL/6 mice. Twelve days later the growing tumours were collected and processed into ˜2 mm² fragments for transplantation into the flank of WT C57/BL/6J mice (8-12 weeks old; Harlan) under full anaesthesia.

The transplanted WT C57/BL/6 mice received 3 intratumor injections over 14 days in a schedule as depicted in FIG. 1B. Thus, on day 2, 7 and 11 after transplantation of the tumors the mice received intratumor injections using 100 μl phosphate buffer saline (PBS), 50 μg of CuMVtt830-VLP alone diluted in a final volume of 100 μl PBS, or 50 μg CuMVtt830-VLP formulated with 4% w/v MCT (40 mg/ml) in a final volume of 100 μl/dose (CuMVtt830-VLP is abbreviated in FIG. 1B as CuMVtt-VLPs).

Tumour growth was followed every two days and measured using callipers according to the formula V=(W×W×L)/2, V=the final tumour volume in mm³, L=tumour length and W=tumour width. Tumours were collected and measured on day 14. Tumours were collected in DMEM medium containing 10% FBS and 1% Penicillin/Streptomycin on ice. Tumour infiltrating lymphocytes (TILs) were collected as following: tumours were dissected into pieces and smashed using 70 μM cell strainer, cells were washed during the process using DMEM medium containing 10% FBS and 1% Penicillin/Streptomycin in falcon tubes 50 ml. Collected cells were added to 15 ml tubes containing 2 ml of 35% Percoll slowly. The tubes were centrifuged at 1800 rpm for 25 min at RT to isolate TILs. TILs were then resuspended in 200 μl PBS, 0.1% BSA and 100 μl was transferred to 96 well plate v-bottom and centrifuged at 1200 rpm for 5 min. Supernatant was discarded, and RBCs were lysed using ACK 500 μl ACK buffer (Sigma-Aldrich) on ice for 2-3 min. TILs were stained with anti-mouse CD16/CD32 (mouse BD Fc block) mAb clone 2.4G2 (BD Bioscience) for 10 min in the dark for 10 min in the dark, centrifuged as described above and stained with PE anti-mouse CD8α mAb clone 53-6.7 (BD Bioscience). Plate was centrifuged at 1200 rpm for 5 min, supernatant was discarded, TTLs were resuspended in PBS, 0.1% BSA and added to 5 ml round-bottom tubes with cell strainer to remove excess tumour debris. Samples were read by FACSCaliber and analysis was done using GraphPad Prism version 8.4.2 (464). Furthermore, CD8⁺ T cell density was measured by dividing the total number of CD8⁺ T cells in each tumor by its volume.

On day 12 whole blood was collected in heparin to assess the percentage of myeloid-derived suppressor cells (MDSCs) in the blood of treated mice, specifically of CD11b^{Hi} Ly6C^{Hi} In detail, blood was collected on day 12 post tumour transplantation from control and treated groups. Incision in mice’ tail vein was performed to collect blood. 150 μl blood was collected in 500 μl 1×PBS containing heparin and kept on ice. Cells were centrifuged at 1200 rpm for 5 min and supernatant was aspirated. RBCs were lysed using 500 μl ACK buffer (Sigma-Aldrich) on ice for 2-3 min. Cells were collected by centrifugation 5 min at 1200 rpm. Supernatant was aspirated and cells were resuspended with 1×PBS containing 0.1% BSA and centrifuged again. Pelleted cells were stained with anti-mouse CD16/CD32 (mouse BD Fc block) mAb clone 2.4G2 (BD Bioscience) for 10 min in the dark, centrifuged as described above and stained with PE anti-mouse CD8α mAb clone 53-6.7 (BD Bioscience) or FITC anti-mouse Ly6C clone HK1.4 (BioLegend) and APC/Cyanine7 anti-mouse CD11b clone M/170 (BioLegend) according to the experiment. Samples were read by FACSCaliber and analysis was done using GraphPad Prism version 8.4.2 (464).

### Results

A significant decrease in the % of CD11bHi Ly6CHi population in the group treated with CuMVtt830-VLP+MCT was observed (FIG. 1C). On days 14 the mice were sacrificed and tumor analysis was performed. The tumor volume mm³ was measured for all mice (FIG. 1D). CuMVtt830-VLP alone showed significant reduction in tumor size when compared to the group treated with PBS. However, the anti-tumor effect was much more pronounced in the group treated with CuMVtt830-VLP formulated with MCT, as described above, when compared to all other groups. The effect of the intratumor treatment on tumor lymphocytic composition, specifically CD8+ T cells, was further studied. It is readily accepted that CD8+ T cells infiltration into the tumor correlates with better prognosis and is an essential piece of evidence for effective immune response (Haanen J B, et al. Melanoma-specific tumor-infiltrating lymphocytes but not circulating melanoma-specific T cells may predict survival in resected advanced-stage melanoma patients. Cancer Immunol Immunother. 2006; 55(4):451-8.4). The composition comprising particles of CuMVtt830-VLP and MCT significantly increased the density of CD8+ T cells infiltration into the tumor when compared to other groups (FIG. 1E). The measured density of CD8+ T cells correlated significantly with tumor volume mm³ (FIG. 1F). We also assessed the density of CD8+ IFN-γ+ T cells in the tumors, the data showed that the group treated with the composition comprising CuMVtt-VLP and MCT showed the strongest cytokine production (FIGS. 1G and H).

Furthermore, intra-cellular cytokine (ICS) staining protocol was performed using specifically anti-CD8α mAb as a surface marker and IFN-γ mAb as an intra-cellular cytokine marker. In detail, 100 μl of the TILs as described above were transferred to sterile 96 well plate flat-bottom for ICS experiment. TILs were incubated with mouse IL-2 (mIL2-Ref: 11271164001-MERCK) 100 U/ml in DMEM medium containing 10% FBS and 1% Penicillin/Streptomycin at 37° C. for 2 days. TILs were washed 3× with DMEM medium containing 10% FBS and 1% Penicillin/Streptomycin and a stimulation cocktail was added 2 μg/ml of Ionomycin, 2 μg/ml of PMA and Brefeldin and Monensin (1:1000)) at 37° C. for 6 h. TILs were washed 3× with DMEM medium to remove the stimulation cocktail and then transferred to 96 well plate v-bottom for staining. TILs were stained with anti-mouse CD16/CD32 (mouse BD Fc block) mAb clone 2.4G2 (BD Bioscience) for 10 min in the dark for 10 min in the dark, centrifuged as described above and stained with PE anti-mouse CD8α mAb clone 53-6.7 (BD Bioscience). The plate was centrifuged at 1200 rpm for 5 min, supernatant was discarded and TILs were fixed using 100 μl of the fixation buffer (BD Cytofix) at 4° C. for 15 min. The plate was centrifuged at 1200 rpm for 5 min, supernatant was discarded, and TILs were washed with 100 μl of 1× diluted permeabilization wash buffer (BioLegend) and centrifuged immediately at 1200 rpm for 5 min, supernatant was discarded. TILs were then stained with APC anti-mouse IFN-γ mAb clone XMG1.2 (MERCK) and PerCP-Cyanine5.5 anti-mouse TNF-α mAb clone MP6-XT22 (BioLegend). Plate was centrifuged at 1200 rpm for 5 min, supernatant was discarded, TILs were resuspended in PBS, 0.1% BSA and added to 5 ml round-bottom tubes with cell strainer to remove excess tumour debris. Samples were read by FACSCaliber and analysis was done using GraphPad Prism version 8.4.2 (464).

Representative FACS plot showing the total number of CD8+ IFN-γ+ producing cells, pre-gated on TILs are shown in FIG. 1I.

### Example 5

### Systemic Effect Induced Upon Intratumor Injections of the Composition Comprising CuMVtt830-VLP and MCT

To study the systemic response of the composition of CuMVtt830-VLP and MCT, the antibody titer induced against CuMVtt830-VLP in the serum of treated mice as a biomarker was measured.

Hereto, serum was collected on day 12 as follows: The mice were warmed up for 15 minutes, 100 μl of whole blood was collected from mice tail in BD Microtainer tubes. Serum was separated by centrifugation at 8000 rpm for 1 minute and stored in −20° C. For determination of total IgG antibody titer against CuMVtt830-VLP, ELISA plates were coated over night with CuMVtt830-VLP at a concentration of 2 μg/ml. Plates were washed with PBS-0.01% Tween and blocked using 100 μl PBS-Casein 0.15% for 2 h. Sera from treated mice were diluted 1/20 initially and a 1/3 dilution chain was performed. Plates were incubated for 1 h at RT. After washing with PBS-0.01% Tween, goat anti-mouse IgG conjugated to Horseradish Peroxidase (HRP) was added 1/1000 and incubated for 1 h at RT. Plates were developed and OD 450 reading was performed.

The groups treated with CuMVtt830-VLP or with cCuMVtt830-VLP formulated with MCT, as described above, had anti-VLP antibodies as expected while no antibodies have been detected in the control group (FIG. 2A and FIG. 2B). The obtained results indicate that intratumor injections of our novel therapy can induce a systemic effect.

### Example 6

### Composition Comprising CuMVtt830-VLP and MCT Decrease the Tumour Mitotic Rate (TMR) as Well as Necrosis

To further study the effect of intratumor injections, a similar experiment as described in Example 4 was performed.

On day 14, the tumors were collected from each group, i.e. the composition of CuMVtt830-VLP and MCT and PBS as control, and the median-sized tumor in each group was kept in 4% paraformaldehyde for 1 day before histological analysis. One full section of each collected tumor was examined after melanin bleach followed by H&E staining. Table 1 shows general characteristics of the collected tumors, specifically: tumor diameter and depth (mm). The composition comprising CuMVtt830-VLP and MCT showed the smallest diameter and depth.

TABLE 1
Tumor labelling and tumor diameter and depth.

| ID | Tumor diameter (mm) | Tumor depth (mm) |
|---|---|---|
| PBS | 20 × 15 | 12 |
| CuMVtt830-VLP + MCT | 10 × 6 | 4 |

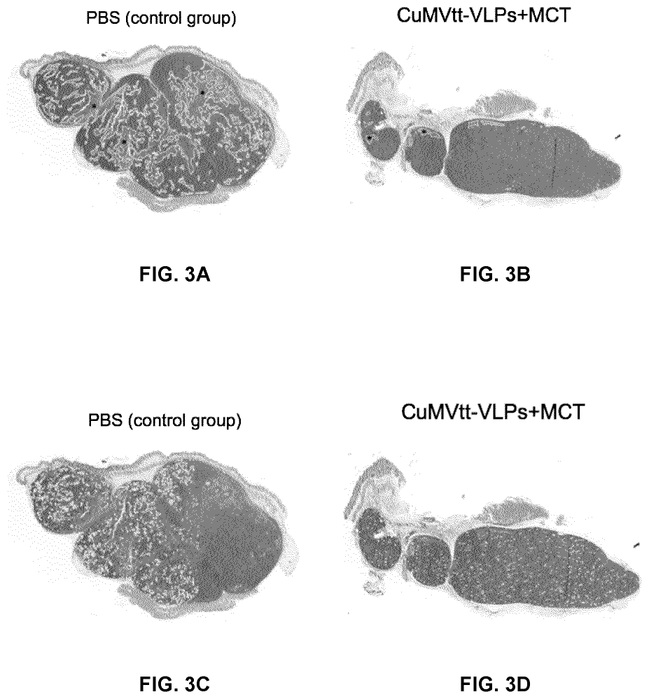

It has been shown earlier in different studies that the prognostic impact of tumor necrosis may represent a paradoxical relationship whereby evidence of increased tumor cell death indicates a more aggressive cancer. However, this relationship can be explained by rapid tumor growth that has outgrown its own blood supply, creating a hypoxic microenvironment and subsequently causing tumor cell death. The presence of necrosis was significantly associated with advanced stage, poor differentiation, venous invasion, and large tumor size (Swinson D E B et al., Lung Cancer. 2002, 37(3):235-40; Pollheimer M J et al., Hum Pathol. 2010, 41(12):1749-57). Tumor-mitotic rate (TMR) is considered an important and independent predictor of survival for melanoma patients. Studies from different centers showed TMR can improve the accuracy of melanoma staging (Azzola M F et al., Cancer. 2003, 97(6):1488-98; Thompson J F et al., J Clin Oncol. 2011, 29(16):2199-205). It has also been shown that TMR probably indicate the depth of the tumor (Basir H R G et al., Clin Cosmet Inv Derm. 2018, 11:125-30). Accordingly, a digital histological assessment was performed and the results indicate a significant decrease in the percentage of necrosis in the group treated with the composition comprising CuMVtt830-VLP and MCT (FIG. 3A, FIG. 3B). This was also the case when comparing the number of mitosis per mm³ (FIG. 3C, FIG. 3D). These findings are listed in Table 2.

TABLE 2
Digital histological assessment of the tumor
mitotic rate (TMR) and tumor necrosis

| | No. of | | | | |
|---|---|---|---|---|---|
| | mitosis | No. of | Total | Total | |
| | in the | mitosis | tumor | tumor | |
| | assessed | per | area | necrosis | % tumor |
| ID | tumor area | mm³ | mm³ | mm³ | necrosis |
| PBS | 2562 | 60.87 | 92.11 | 27.77 | 30.15 |
| CuMVtt830- | 762 | 47.12 | 16.5 | 0.33 | 1.98 |
| VLP + MCT | | | | | |

### Example 7

### Composition Comprising CuMVtt830-VLP and MCT Increases CD8⁺ T Cells

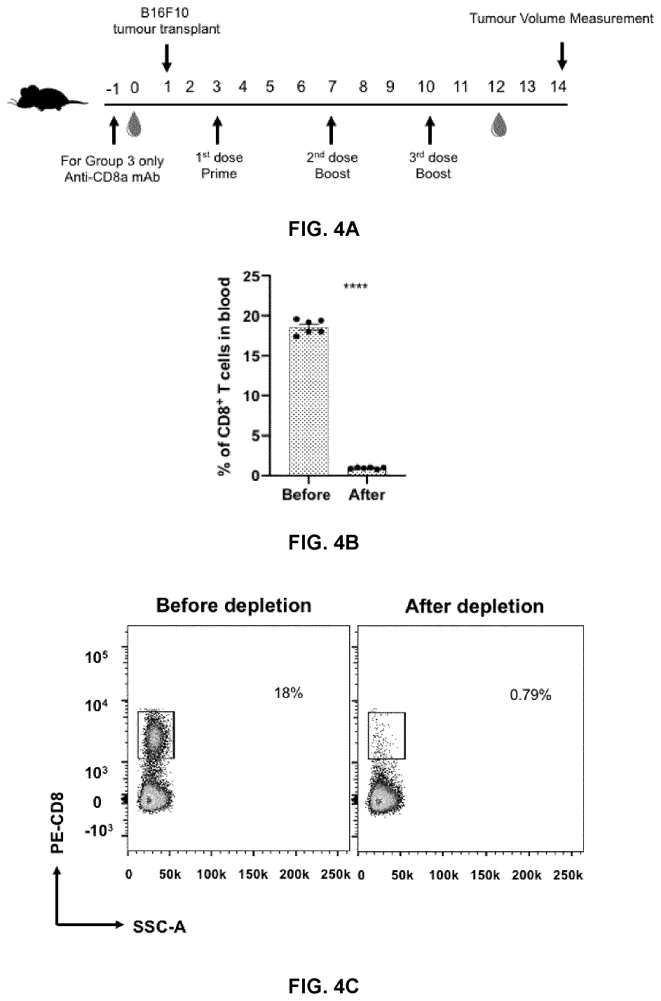

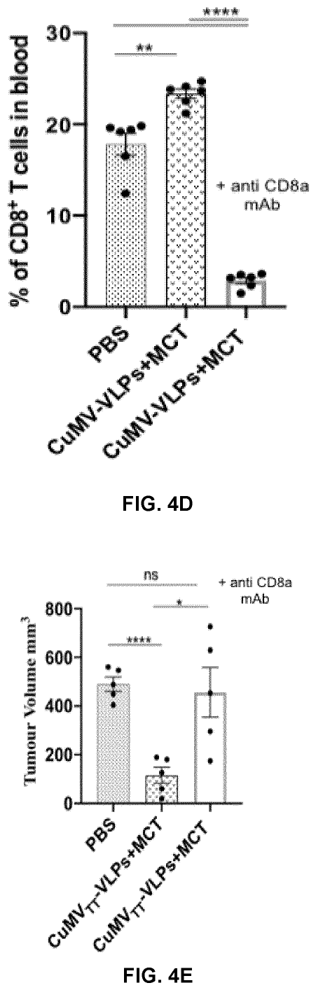

To study the role of CD8⁺ T cells in the mechanism of action of intratumor injections of compositions comprising CuMVtt830-VLP and MCT, we carried out an experiment as summarized in FIG. 4A with three groups of mice as follows:

| Group 1 | Group 2 | Group 3 |
|---|---|---|
| PBS | CuMVtt830-VLP + MCT | CuMVtt830-VLP + MCT |
| | 50 μg/dose + 100 μl/dose* | 50 μg/dose + 100 μl/dose* + |
| | | anti-CD8a mAb |
| | | 10 μg/mouse every 2 days |

*prepared as described using 4% MCT (40 mg/ml; Allergy Therapeutics Ltd. Worthing, UK)

In group 3, CD8⁺ T cells were depleted from C57BL/6 blood by using anti-CD8α mAb (Clone 53-6.7). Briefly, 10 ug/dose of anti-CD8α mAb was administered i.v. two days before tumour transplantation and later every 2 days. CD8⁺ T cells in the blood were checked before tumour transplantation and on day 12 post tumour transplantation. Tumor bearing mice were treated with PBS or with the composition comprising CuMVtt830-VLP and MCT on days 2, 7 and 11 (with/without CD8⁺ T cells depletion). Mice were treated 3 times over 14 days. Depletion of CD8⁺ T cells showed ˜95% efficiency (FIG. 4B and FIG. 4C). On day 12, the frequency of CD8⁺ T cells was checked again and the results indicate that intratumor injection of CuMVtt830-VLP+MCT increase % of CD8⁺ T cells in the blood in group 2 when compared to the control group or group 3 (FIG. 4D). This can be used as a prognostic marker. On day 14, tumors were collected and tumor volume was measured. Results show that the depleting CD8⁺ T cells hinder anti-tumor efficacy of the treatment, indicating the essential role of CD8⁺ T cells (FIG. 4E).

### Example 8

### Composition Comprising CuMVtt830-VLP and MCT Induces Abscopal Effect Upon Intratumor Injection

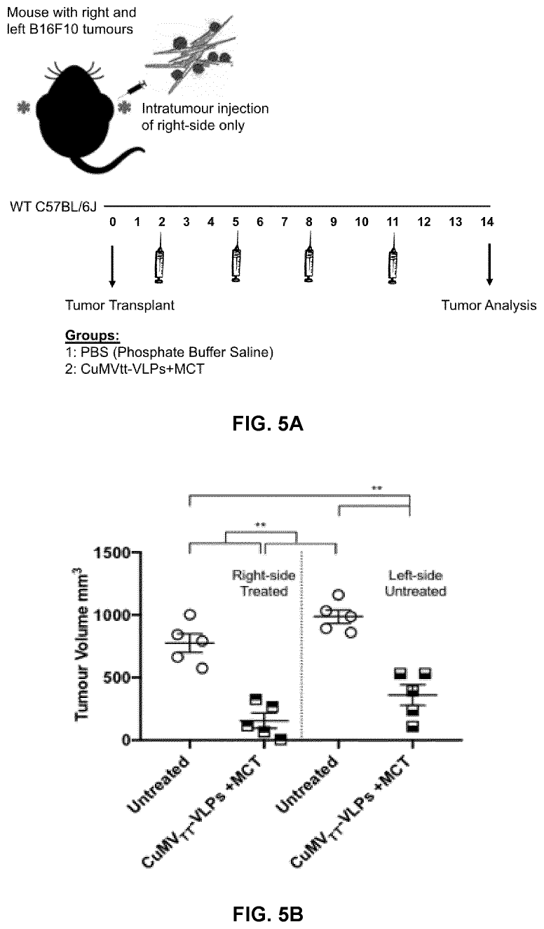

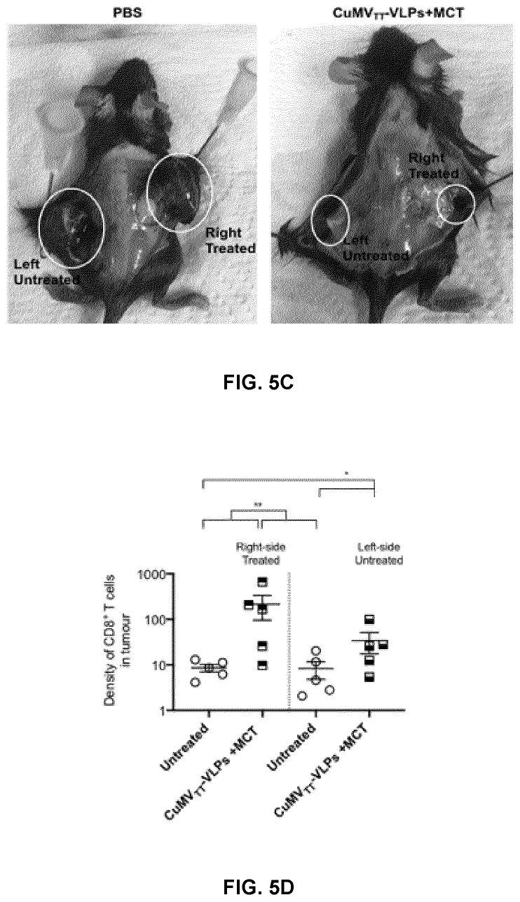

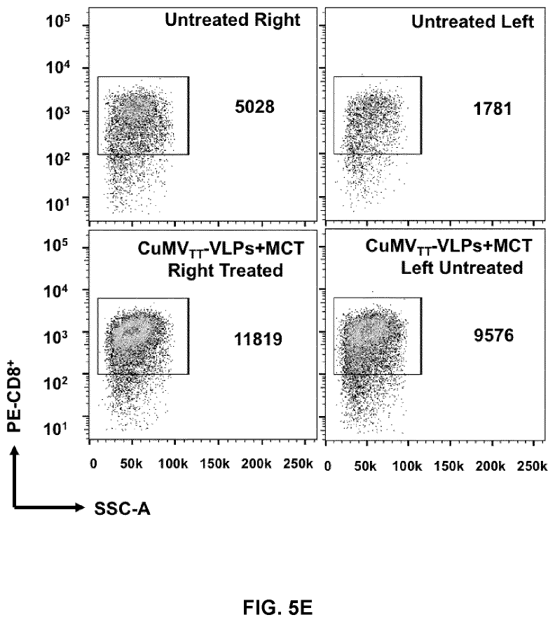

To study the abscopal effect of the intratumor treatment, we have established an experiment as illustrated in FIG. 5A. Two groups have been used in this experiment, a control group PBS and a treated group using CuMV_{TT}-VLPs formulated with MCT (CuMV_{TT}-VLPs 50 ug/dose and MCT 4% at 100 ul/dose). Right transplanted tumors were treated by intratumor injections while the left tumor was kept untreated. As the tumor burden is higher in this experiment, we decided to increase the intratumor injections to twice a week. We have also tested the impact of anti-PD1 (i.v. injections) when combined with CuMV_{TT}-VLPs formulated with MCT. However, the results did not show any additional anti-tumor efficacy (data not shown) which could be explained with the fact that B16F10 melanoma tumor is non-responsive to anti-PD1 treatment (3). The obtained results revealed that intratumor injection of the combination of CuMV_{TT}-VLPs+MCT can generate anti-tumor response in the injected tumor as well as abscopal effect (FIG. 5B and Fig. C). Such finding has also been confirmed when measuring the density of CD8⁺ T cells in the treated and untreated tumors as shown in (FIG. 5D and FIG. 5E). These findings correlate with the results obtained in Example 2 which indicates that a systemic effect can be induced with intratumor injection.

### Example 9

### Anti-Tumor Efficacy Upon Intratumor Injection Using Different VLPs Formulated with MCT

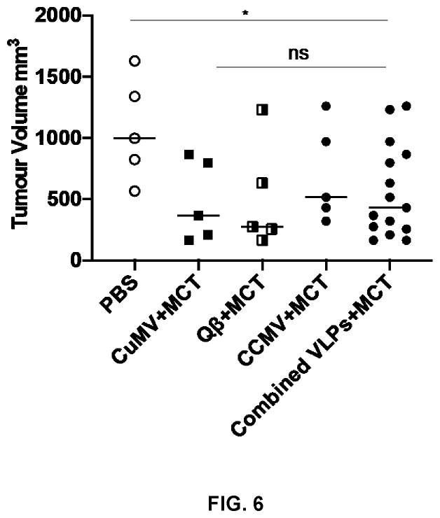

Different inventive compositions comprising different VLPs were prepared and tested for their anti-tumor effect. Thus, a similar experiment as described in Example 4 was performed using the following groups: PBS (control), CuMV-VLPs formulated with MCT, Qβ-VLPs formulated with MCT or CCMV-VLPs formulated with MCT. The tumors were treated intratumorally 3 times over 14 days. Tumors were collected on day 14 and tumor volume was measured as described earlier. The results indicate that formulating different VLPs with MCT can induce anti-tumor effect in B16F10 transplanted melanoma model (FIG. 6).

### Example 10

### Effective Anti-Tumor Response and Long-Term Protection of CuMVtt-VLPs Formulated with MCT Adjuvant in Equine Melanoma Model

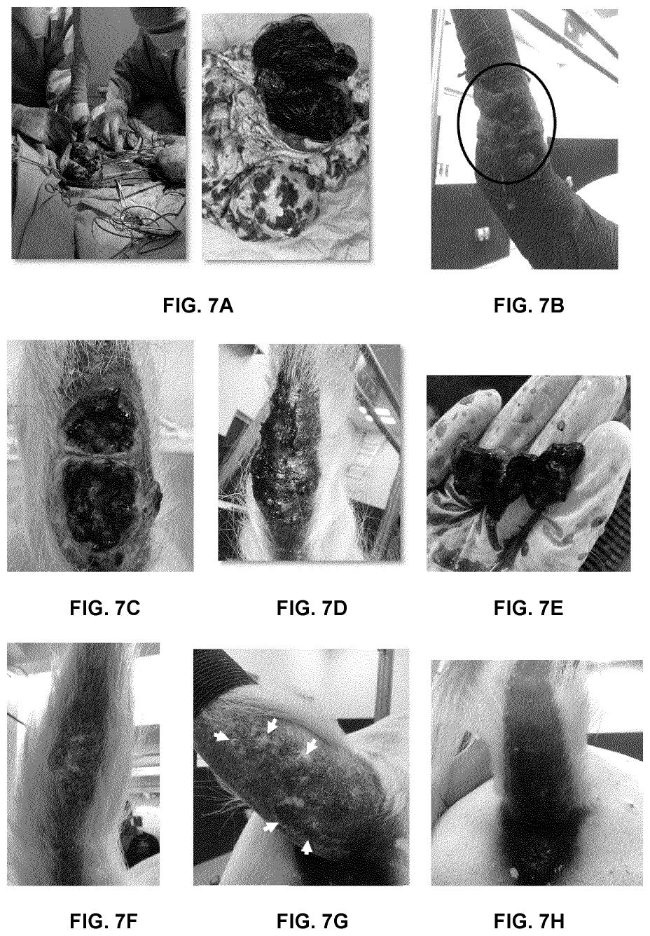

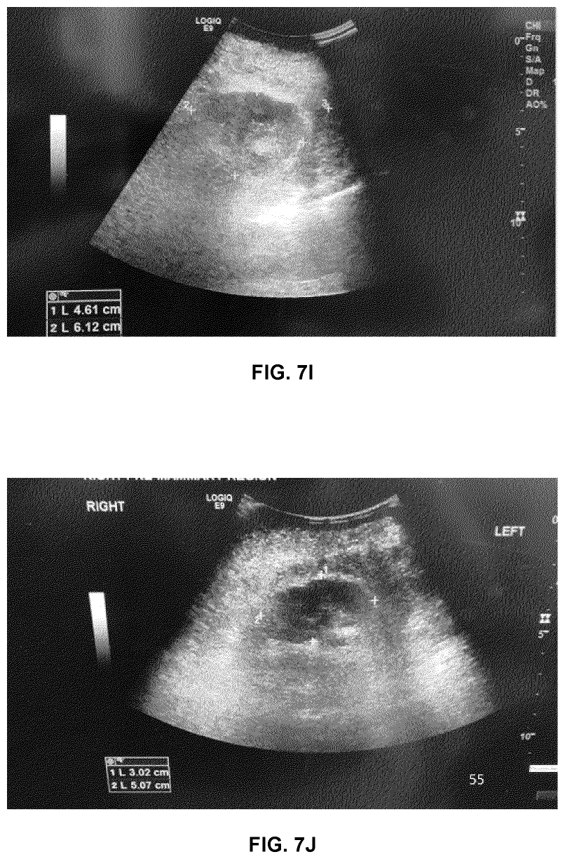

The efficacy of the novel treatment with a composition comprising particles of CuMVtt830-VLP and MCT was tested in equine melanoma models. Two privately owned Arabic grey/white horses with heavy melanoma metastases, already scheduled to be euthanized, were treated with the composition comprising the CuMVtt830-VLP and MCT formulation. The first horse is a mature (Federation Equestre Internationale) FEI endurance grey stallion that suffered from melanoma infiltrating the peripenile region and several external melanoma lesions on the tail. The internal melanoma mass was surgically removed and the penis had to be amputated due to a large preputial mass (FIG. 7A). One week later, the intratumor treatment of the external melanoma lesions started, total 4 injections (with 10 days interval) were administered using pressure syringes. The induced anti-tumor response was followed weekly, in addition to measuring temperature, complete blood count (CBC) and overall health assessment. The consistency of the hard-external melanoma lesions was changing over the treatment period, becoming softer and leaking dark secretion. The recorded adverse effects included mild itching, but there was no fever and normal CBC. Thus, the horse responded well to the treatment and the tumor reduced in size indicating efficacy of the novel treatment in eliminating large melanoma lesions in a grey horse (FIG. 7B-F). The horse was followed for a year with no signs of relapse. Additionally, a distant (“abscopal”) effect was observed in untreated melanoma nodules at the upper part of the tail (FIGS. 7G and H). The second horse case is a pregnant grey Arabian mare, diagnosed with a mammary gland lump. Ultrasound-guided intratumor injection of the novel immune-enhancer (total 2 injections so far) were administered. No side-effects were reported and a notable reduction in the internal mass has been seen as shown in FIGS. 7I and J. Treatment could not be continued as the horse was scheduled for euthanasia.

1. A composition comprising particles, wherein each particle comprises, preferably consists of,
(a) at least one virus-like particle (VLP), preferably a recombinant VLP, and
(b) at least one microcrystalline tyrosine, preferably microcrystalline L-tyrosine, wherein said microcrystalline tyrosine has a median particle size of and between 0.1 μm and 50 μm, as preferably determined by Flow Particle Image Analyser (FPIA) and further preferably as described in Example 2; and
wherein said VLP does not comprise a tumor-associated antigen or a tumor-specific antigen covalently linked to said VLP, and wherein further preferably said VLP does not comprise an antigen covalently linked to said VLP.

2. The composition of claim 1, wherein said microcrystalline tyrosine has a maximum particle size of 750 μm, wherein preferably said microcrystalline tyrosine has a maximum particle size of 150 μm.

3. The composition of any one of the preceding claims, wherein said VLP is adsorbed on said microcrystalline tyrosine.

4. The composition of any one of the preceding claims, wherein said composition is an aqueous composition, wherein preferably the concentration of said microcrystalline tyrosine, preferably said microcrystalline L-tyrosine, is 0.5% to 10% (weight tyrosine/volume solution), and wherein preferably the concentration of said microcrystalline tyrosine, preferably said microcrystalline L-tyrosine, is 1% to 7.5% (weight tyrosine/volume solution).

5. The composition of any one of the preceding claims, wherein said VLP is a modified VLP, wherein said modified VLP comprises, preferably consists of, at least one modified VLP polypeptide, wherein said modified VLP polypeptide comprises,
(a) a VLP polypeptide, and
(b) a T helper cell epitope,
wherein said VLP polypeptide comprises, or preferably consists of,
(i) an amino acid sequence of a coat protein of a virus, preferably an amino acid sequence of a coat protein of a plant virus; or
(ii) a mutated amino acid sequence, wherein said mutated amino acid sequence and said coat protein of a virus show a sequence identity of at least 90%, preferably of at least 95%, further preferably of at least 98% and again more preferably of at least 99%.

6. The composition of any one of the preceding claims, wherein said VLP is derived from a plant virus or is a VLP of an RNA bacteriophage, wherein preferably said plant virus is selected from the group consisting of cucumber mosaic virus (CuMV), cowpea chlorotic mottle virus (CCMV) and tobacco mosaic virus (TMV), and wherein preferably said VLP of an RNA bacteriophage is a VLP of an RNA bacteriophage Qbeta or a VLP of an RNA bacteriophage AP205.

7. The composition of any one of the preceding claims, wherein said VLP is a modified VLP of cucumber mosaic virus (CuMV), wherein said modified VLP of CuMV comprises, preferably consists of, at least one modified CuMV polypeptide, wherein said modified CuMV polypeptide comprises, or preferably consists of,
(c) a CuMV polypeptide, and
(d) a T helper cell epitope; and
wherein said CuMV polypeptide comprises, or preferably consists of,
(ii) an amino acid sequence of a coat protein of CuMV; or
(ii) a mutated amino acid sequence, wherein said mutated amino acid sequence and said coat protein of CuMV show a sequence identity of at least 90%, preferably of at least 95%, further preferably of at least 98% and again more preferably of at least 99%.

8. The composition of claim 7, wherein said CuMV polypeptide is a coat protein of CuMV or an amino acid sequence having a sequence identity of at least 90%, preferably 95% with SEQ ID NO:1.

9. The composition of claim 7 or claim 8, wherein said T helper cell epitope replaces a N-terminal region of said CuMV polypeptide, and wherein said N-terminal region of said CuMV polypeptide corresponds to amino acids 2-12 of SEQ ID NO:1, and wherein preferably said Th cell epitope is derived from tetanus toxin or is a PADRE sequence.

10. The composition of any one of the claims 5 to 9, wherein said Th cell epitope comprises the amino acid sequence of SEQ ID NO:3 or SEQ ID NO:4.

11. The composition for use of any one of the claims 7 to 10, wherein said modified CMV polypeptide comprises, preferably consists of, an amino acid sequence of SEQ ID NO:11 or SEQ ID NO:12.

12. The composition of any one of the preceding claims, wherein said VLP, preferably said recombinant VLP, comprises an immunostimulatory substance packaged in said VLP, wherein preferably said immunostimulatory substance is a toll-like receptor activating substance, and wherein further preferably said toll-like receptor activating substance is an immunostimulatory nucleic acid, and wherein again further preferably said immunostimulatory nucleic acid is a ssRNA.

13. A composition of any one of the claims 1 to 12 for use in a method of treating a solid tumor in a patient, wherein said patient is a human patient or an animal patient, and wherein preferably said animal patient is selected from an equine animal, preferably a horse, a dog, or a cat, and wherein further preferably said animal patient is a horse.

14. The composition for use of claim 13, wherein said method of treating a solid cancer in a patient is a method of intratumorally treating said solid cancer in a patient.

15. The composition for use of claim 13 or claim 14, wherein said solid cancer is selected from breast cancer, prostate cancer, colorectal cancer, lung cancer and melanoma cancer, preferably melanoma cancer.


SEQUENCE LISTING (16 sequences)

SEQ ID NO: 1 - protein, 218 aa, Cucumber mosaic virus
MDKSESTSAGRSRRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 2 - protein, 27 aa, synthetic construct
DKSESTSAGRSRRRRPRRGSRSAPSSA

SEQ ID NO: 3 - protein, 14 aa, synthetic construct
QYIKANSKFIGITE

SEQ ID NO: 4 - protein, 13 aa, synthetic construct
AKFVAAWTLKAAA

SEQ ID NO: 5 - protein, 21 aa, synthetic construct
FNNFTVSFWLRVPKVSASHLE

SEQ ID NO: 6 - protein, 13 aa, synthetic construct
PKYVKQNTLKLAT

SEQ ID NO: 7 - protein, 20 aa, synthetic construct
PHHTALRQAILCWGELMTLA

SEQ ID NO: 8 - protein, 21 aa, synthetic construct
DIEKKIAKMEKASSVFNVVNS

SEQ ID NO: 9 - protein, 16 aa, synthetic construct
YSGPLKAEIAQRLEDV

SEQ ID NO: 10 - protein, 217 aa, Cucumber mosaic virus
DKSESTSAGRNRRRRPRRGSRSASSSADANFRVLSQQLSRLNKTLAAGRPTINHPTFVGSERCKPGYTFSSITLKPPKIDRGSYYGKRLLLPDSVTEFDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDIEHQRIPTSGVLPV

SEQ ID NO: 11 - protein, 222 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 12 - protein, 220 aa, synthetic construct
MAKFVAAWTLKAAARRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 13 - protein, 218 aa, Cucumber mosaic virus
MDKSESPNASRTSRRRRPRRGSRSASGADAGLRALTQQMLKLNKTLAIGRPTLNHPTFVGSASCKPGYTFTSITLKPPEIEKGSYFGRRLSLPDSVTDYDKKLVSRIQIRINPLPKFDSTVWVTVRKVPSSSDLSVATISAMFGDGNSPVLVYQYTASGVQANNKLLYDLSEMRADIGDMRKYAVLVYSKDDKLEEDEIVLHVDVEHQRIPISRMLPT

SEQ ID NO: 14 - protein, 132 aa, Bacteriophage Q-beta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 15 - protein, 329 aa, Bacteriophage Q-beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 16 - protein, 131 aa, Bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA